(19) United States
(12) Patent Application Publication
(10) Pub. No.: US 2026/0096841 A1
(43) Pub. Date: Apr. 9, 2026

(54) ELECTROPORATION SYSTEM

(71) Applicant: Argá Medtech SA, Epalinges (CH)

(72) Inventors: Melvin LORENZO, San Diego, CA (US); Mark ZURCHER, Encinitas, CA (US); Micah A. LEE, Escondido, CA (US); Alexander Kyle LOPEZ, San Diego, CA (US); Randell L. WERNETH, San Diego, CA (US); Marshall SHERMAN, Cardiff, CA (US); Graydon Ernest BEATTY, San Diego, CA (US); R. Maxwell FLAHERTY, Topsfield, MA (US); J. Christopher FLAHERTY, Nottingham, NH (US)

(73) Assignee: Argá Medtech SA, Epalinges (CH)

(21) Appl. No.: 19/115,161

(22) PCT Filed: Sep. 27, 2023
(86) PCT No.: PCT/IB2023/059645

§ 371 Date: Mar. 25, 2025

Related U.S. Application Data
(60) Provisional application No. 63/410,391, filed on Sep. 27, 2022.

(51) Int. Cl. A61B 18/00

(52) U.S. Cl. CPC A61B 18/00; A61B 2018/00613; A61B 2018/00761

(57) ABSTRACT

Provided herein are systems, devices, and methods for delivering electroporation energy to treat target tissue. The system includes a generator for providing an electroporation waveform. The generator includes a signal generator for generating the electroporation waveform and a controller for providing signaling that causes the signal generator to generate the electroporation waveform. The system further includes a catheter including at least one catheter electrode. The signal generator provides the electroporation waveform to the at least one catheter electrode. The electroporation waveform includes a plurality of energy pulses, and each energy pulse is separated by an inter-pulse delay period.

### CROSS REFERENCE TO RELATED APPLICATIONS

The present application claims priority to U.S. Provisional Patent Application Ser. No. 63/410,391, filed Sep. 27, 2022, entitled “A Controller for an Electroporation Apparatus”, which is incorporated by reference.

The present application, while not claiming priority to, may be related to: U.S. patent application Ser. No. 17/686,001, filed Mar. 3, 2022, entitled “Ablation Equipment to Treat Target Regions of Tissue in Organs”; U.S. patent application Ser. No. 17/686,027, filed Mar. 3, 2022, entitled “Ablation Equipment to Treat Target Regions of Tissue in Organs”; U.S. patent application Ser. No. 17/939,465, filed Sep. 7, 2022, entitled “Systems, Methods and Devices for Non-Thermal Ablation of Target Tissue”; U.S. patent application Ser. No. 18/001,041, filed Dec. 7, 2022, entitled “Ablation Equipment to Treat Target Regions of Tissue in Organs”; U.S. patent application Ser. No. 18/258,466, filed Jun. 20, 2023, entitled “Electronic Apparatus for Delivering Coherent Sine Burst Irreversible Electroporation Energy to a Biological Tissue”; and U.S. patent application Ser. No. 18/338,135, filed Jun. 20, 2023, entitled “Power Unit for Delivering Coherent Sine Burst Irreversible Electroporation Energy to a Biological Tissue”; each of which is hereby incorporated by reference.

### FIELD OF THE INVENTIVE CONCEPTS

The present disclosure relates to an electroporation system, an electroporation controller, a method of controlling an electroporation system and a computer program product comprising computer program code configured to cause a controller to perform the method.

### BACKGROUND

Tissue ablation is used in numerous medical procedures to treat a patient. Ablation can be performed to remove or denature undesired tissue such as cardiac cells. The ablation can be performed by passing energy, such as electrical energy, through one or more electrodes and causing tissue death where the electrodes are in contact with tissue. Ablation procedures can be performed on patients with any cardiac arrhythmia such as atrial fibrillation (AF) by ablating tissue in the heart.

### SUMMARY

According to an aspect of the present inventive concepts, a system for delivering electroporation energy to target tissue to be treated comprises a generator configured to provide an electroporation waveform, and the generator includes a signal generator configured to generate the electroporation waveform and a controller configured to provide signaling configured to cause the signal generator to generate the electroporation waveform. The system further comprises a catheter including at least one catheter electrode. The signal generator is configured to provide the electroporation waveform to the at least one catheter electrode. The electroporation waveform comprises a plurality of energy pulses, and each energy pulse is separated by an inter-pulse delay period.

In some embodiments, the inter-pulse delay period comprises a first delay period and a second delay period, and the first delay period comprises a fixed duration between each of the plurality of energy pulses and the second delay period comprises a variable duration between each of the plurality of energy pulses. Each variable duration can comprise a positive duration, a negative duration, or both. The inter-pulse delay period can comprise a first inter-pulse delay period between a first energy pulse of the plurality of energy pulses and a second energy pulse of the plurality of energy pulses, and a second inter-pulse delay period between the second energy pulse and a third energy pulse of the plurality of energy pulses, and the first inter-pulse delay period can comprise a first variable duration and the second inter-pulse delay period can comprise a second variable duration. The first variable delay period can comprise a positive duration and the second variable delay period can comprise a negative duration. The duration of the first variable duration can be equal to the absolute value of the duration of the second variable duration. The variable duration can comprise a duration based on a pseudo-random number.

In some embodiments, the inter-pulse delay period comprises a variable duration between each energy pulse. The variable duration can comprise a duration based on a pseudo-random number. The variable duration can be configured to reduce harmonics created by delivery of the energy pulses. The variable duration can be configured to reduce the harmonics by at least 10 dB.

In some embodiments, the electroporation waveform further comprises a cycle length, and the cycle length comprises the duration from the start of a first energy pulse of the plurality of energy pulses to the start of the subsequent energy pulse of the plurality of energy pulses. The cycle length can be configured to minimize microbubble formation. The cycle length can comprise a duration of at least 30 ms.

In some embodiments, the inter-pulse delay period comprises a duration of at least 1 ms.

In some embodiments, the inter-pulse delay period comprises a duration of no more than 2000 ms.

In some embodiments, the controller comprises a processor and a memory storage component coupled to the processor, and the memory storage component stores instructions for the processor to perform an algorithm. The algorithm can be configured to determine one or more parameters of the electroporation waveform. The algorithm can comprise one or more biases. The one or more biases can be configured to determine the one or more parameters of the electroporation waveform such that the electroporation waveform tends toward: a particular frequency range; a particular ratio of bipolar-to-unipolar energy delivery; a particular phase difference between included sine waves; a particular voltage or range of voltages; a particular delay between energy deliveries such as a particular inter-pulse delay; and combinations thereof.

In some embodiments, the at least one catheter electrode comprises a first set of non-neighboring catheter electrodes and a second set of non-neighboring catheter electrodes. A first catheter electrode of the second set of non-neighboring catheter electrodes can be positioned between a first catheter electrode and a second catheter electrode of the first set of non-neighboring catheter electrodes. A first energy pulse of the plurality of energy pulses can be provided to the first set of non-neighboring catheter electrodes and a second energy pulse of the plurality of energy pulses can be provided to the second set of non-neighboring catheter electrodes. A third energy pulse of the plurality of energy pulses can be provided to the first set of non-neighboring catheter electrodes. The generator can be configured to provide the electroporation waveform in a bipolar arrangement.

In some embodiments, the at least one catheter electrode comprises multiple electrodes, and the multiple electrodes comprise a first catheter electrode and a set of at least two additional catheter electrodes. The signal generator can be configured to provide the electroporation waveform to the first catheter electrode and the set of at least two additional catheter electrodes. The generator can be configured to provide the electroporation waveform in a bipolar arrangement to the first catheter electrode and the at least two additional catheter electrodes. Each electrode of the multiple electrodes can comprise a similar surface area. Each electrode of the multiple electrodes can be equally spaced from each neighboring electrode.

In some embodiments, the system further comprises one or more external electrodes, and the signal generator is configured to provide the electroporation waveform to the at least one catheter electrode and the one or more external electrodes. The one or more external electrodes can comprise at least two external electrodes, and each of the at least two external electrodes can be individually selectable such that the electroporation waveform can be provided to the at least one catheter electrode and any of the at least two external electrodes. The controller can be further configured to select one or more electrodes of the at least two external electrodes to provide the electroporation waveform, such that target tissue to be treated by the delivery of the electroporation waveform can be located relatively between the at least one catheter electrode and the one or more selected external electrodes.

In some embodiments, the at least one catheter electrode comprises multiple catheter electrodes, and the generator is configured to provide the electroporation waveform in a bipolar arrangement between two or more of the multiple catheter electrodes.

In some embodiments, the system further comprises one or more external electrodes, and the electroporation waveform is configured to be delivered in a unipolar arrangement to the at least one catheter electrode and the one or more external electrodes. The electroporation waveform can be configured to be delivered in both a unipolar arrangement and a bipolar arrangement. The electroporation waveform can comprise a first signal comprising a first sine wave and a second signal comprising a second sine wave, and a third signal comprising a combined reference of the first sine wave and the second sine wave, and the first sine wave and the second sine wave can comprise a phase offset, and the first signal can be configured to be provided to a first electrode of the at least one catheter electrodes, the second signal can be configured to be provided to a second electrode of the at least one catheter electrodes, and the third signal can be configured to be provided to at least one of the one or more external electrodes. The electroporation waveform can be provided in a unipolar arrangement when the phase offset of the first and second signals is 0°. The electroporation waveform can be provided in both a unipolar arrangement and a bipolar arrangement when the phase offset of the first and second signals is greater than 0°and no more than 180°. The relative strength of the unipolar energy delivery can be configured to vary relative to the strength of the bipolar energy delivery based on the phase angle.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic example of a system comprising a catheter and a generator for providing electroporation, consistent with the present inventive concepts.

FIG. 2 illustrates an example of a portion of an electroporation waveform, consistent with the present inventive concepts.

FIG. 3 illustrates an example of a portion of an electroporation waveform, consistent with the present inventive concepts.

FIGS. 3A-D illustrate graphs of the harmonics of various frequencies under varying conditions, consistent with the present inventive concepts.

FIGS. 4 and 4A illustrate graphs of experimental results and a portion of an electroporation waveform, respectively, consistent with the present inventive concepts.

FIG. 5 illustrates a plot of stimulation strength versus duration, consistent with the present inventive concepts.

FIG. 6 illustrates an example of an electroporation waveform comprising compensation signals, consistent with the present inventive concepts.

FIG. 7 illustrates an example of an electroporation waveform comprising compensation signals, consistent with the present inventive concepts.

FIG. 8 illustrates an example of an electroporation waveform comprising a concurrent compensation signal, consistent with the present inventive concepts.

FIGS. 9A and 9B illustrate an anatomic side view of a catheter comprising multiple electrodes positioned proximate tissue and a representation of the effective electric field generated by the electrodes, respectively, consistent with the present inventive concepts.

FIGS. 10A-C illustrate anatomic side views of a catheter comprising multiple electrodes positioned proximate tissue and a representation of the effective electric field generated by the electrodes, respectively, consistent with the present inventive concepts.

FIGS. 11A and 11B illustrate representations of the damage to tissue caused by various forms of electroporation, consistent with the present inventive concepts.

FIG. 12 illustrates a side view of a catheter including a plurality of electrodes, consistent with the present inventive concepts.

FIG. 13 illustrates a perspective view of a catheter including a curved array of electrodes, consistent with the present inventive concepts.

FIG. 14 illustrates a perspective view of a catheter including an expandable array of electrodes, consistent with the present inventive concepts.

FIG. 15 illustrates a visual representation of a method of delivering electroporation therapy, consistent with the present inventive concepts.

FIGS. 16A-G illustrate two anatomic representations and a graph of lesion depth, as well as two representations of damage to tissue and graphs of ablation parameters, respectively, consistent with the present inventive concepts.

FIG. 17 illustrates a schematic view of a system for performing unipolar and/or phased-combination energy delivery with multiple external patch electrodes, consistent with the present inventive concepts.

FIGS. 17A-E illustrate sectional views of the setup of a finite element analysis and the results of the analysis, consistent with the present inventive concepts.

FIGS. 18A-C illustrate a graph of a cell membrane potential during an action potential, and examples of various pulse timing methodologies, respectively, consistent with the present inventive concepts.

FIGS. 19A-C illustrate diagrams of various energy delivery modalities, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent”versus “directly adjacent,”etc.).

It will be further understood that when a first element is referred to as being “in”, “on” and/or “within” a second element, the first element can be positioned: within an internal space of the second element, within a portion of the second element (e.g., within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g., a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can”operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g., efficacious therapy) and/or to prevent or otherwise reduce (hereinafter “prevent”) an undesired event (e.g., a device and/or clinical adverse event). In some embodiments, a system parameter is maintained above a first threshold (e.g., above a first temperature threshold to cause a desired therapeutic effect to tissue) and below a second threshold (e.g., below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise perform a treatment on tissue (e.g., a functional element configured as a treatment element). Alternatively or additionally, a functional element (e.g., a functional element comprising a sensor) can be configured to record one or more parameters, such as a patient physiologic parameter; a patient anatomical parameter (e.g., a tissue geometry parameter); a patient environment parameter; and/or a system parameter. In some embodiments, a sensor or other functional element is configured to perform a diagnostic function (e.g., to gather data used to perform a diagnosis). In some embodiments, a functional element is configured to perform a therapeutic function (e.g., to deliver therapeutic energy and/or a therapeutic agent). In some embodiments, a functional element comprises one or more elements constructed and arranged to perform a function selected from the group consisting of: deliver energy; extract energy (e.g., to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g., based on the size of the electrode). In some configurations, a transducer converts an electrical signal into any output, such as: light (e.g., a transducer comprising a light emitting diode or light bulb), sound (e.g., a transducer comprising a piezo crystal configured to deliver ultrasound energy); pressure (e.g., an applied pressure or force); heat energy; cryogenic energy; chemical energy; mechanical energy (e.g., a transducer comprising a motor or a solenoid); magnetic energy; and/or a different electrical signal (e.g., different than the input signal to the transducer). Alternatively or additionally, a transducer can convert a physical quantity (e.g., variations in a physical quantity) into an electrical signal. A transducer can include any component that delivers energy and/or an agent to tissue, such as a transducer configured to deliver one or more of: electrical energy to tissue (e.g., a transducer comprising one or more electrodes); light energy to tissue (e.g., a transducer comprising a laser, light emitting diode and/or optical component such as a lens or prism); mechanical energy to tissue (e.g., a transducer comprising a tissue manipulating element); sound energy to tissue (e.g., a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Tissue ablation is used in numerous medical procedures to treat one or more medical conditions of patient. Ablation can be performed to remove or denature undesired tissue such as cardiac cells associated with an arrhythmia. The ablation can be performed by passing energy, such as electrical energy, through one or more electrodes and causing tissue death at locations proximate the electrodes (e.g., where heat generated by the application of energy is sufficient to cause cell death, and/or where the electric field generated is sufficient to irreversibly electroporate the tissue). Ablation procedures can be performed on heart tissue of patients with any form of cardiac arrhythmia, such as atrial fibrillation (AF).

Radiofrequency ablation (RFA) is a medical procedure in which tissue which is part of the electrical conduction system of the heart, tumor tissue, and/or other dysfunctional tissue is ablated using the heat generated from delivery of alternating current. Typical frequencies of the alternating current in this context may be considered to be from 350 kHz to 500 kHz.

Particularly, in these types of ablation procedures, an energy delivery device, such as a catheter or other probe with one or more electrodes, is inserted proximate target tissue to cause destruction of a target region of the cardiac tissue through the application of thermal energy. In fact, electrical induced thermal ablation, such as RFA, can be used to effectively and continuously locally ablate a tissue site as the energy delivery device is placed on the tissue surface. Although RFA can effectively ablate volumes of target tissue, there are limitations to this thermal technique. One often cited problem using this procedure during cardiac ablation involves heat sink, a process whereby one aspect can include blood flow whereas the heat generated on the ablation element will be removed/dissipated by the cooler blood flow over the element. This heat dissipation effect can change (e.g., undesirably reduce) both the shape and the maximum volume of the tissue being ablated.

More recently, to ablate heart and/or other organ tissue, Pulsed Electric Fields (PEF) have been used as an alternative to the above-mentioned RFA. Pulsed Electric Fields (PEF) refer to the application of intermittent, high-intensity electric fields for short periods of time (e.g., microseconds or nanoseconds), which results in cellular electroporation of tissue. Electroporation is a process whereby an applied electric field (i.e. PEF) results in the formation of pores in cell membranes. Pore formation leads to permeabilization, which can be reversible or irreversible, depending upon the parameters of the applied PEF.

In reversible electroporation, the electroporated cells remain viable. This approach underlies the basis of electrochemotherapy and gene electrotransfer. In contrast, with IRreversible Electroporation (IRE), cells and tissues are made non-viable because the technique induces programmed cell death cascade activation.

IRE is a well-established treatment for solid tumors, however, IRE may also be useful in cardiology, particularly for cardiac ablation, especially given the limitations of current thermal based approaches.

When used to ablate cardiac tissue, IRreversible Electroporation (IRE) involves the application of electrical pulses to targeted tissue for a duration in the range of microseconds to nanoseconds that can lead to non-thermally produced defects in the cell membrane that are nanoscale in size. These defects can lead to a disruption of homeostasis of the cell membrane, thereby causing irreversible cell membrane permeabilization which induces cell death, without significantly raising the temperature of the tissue ablation zone.

In some embodiments, the systems, devices and methods of the present inventive concepts are configured to avoid raising the temperature of tissue proximate the tissue ablation zone to a maximum increase of no more than 13° C., such as no more than 10° C., 7° C., or 4° C., and/or to avoid raising the temperature of tissue proximate the tissue ablation zone to a maximum of no more than 50° C., such as a maximum of no more than 47° C. or 44° C.

The present application relates to providing an electroporation waveform that comprises a plurality of energy pulses each comprising sinewave signals, where the energy pulses are separated by inter-pulse delay periods. By providing for inter-pulse delay periods, as well as other delay periods described herein, it is possible to successfully cause targeted cell death, while avoiding undesirable heating at a target treatment site, while avoiding or at least reducing the formation of microbubbles, or both.

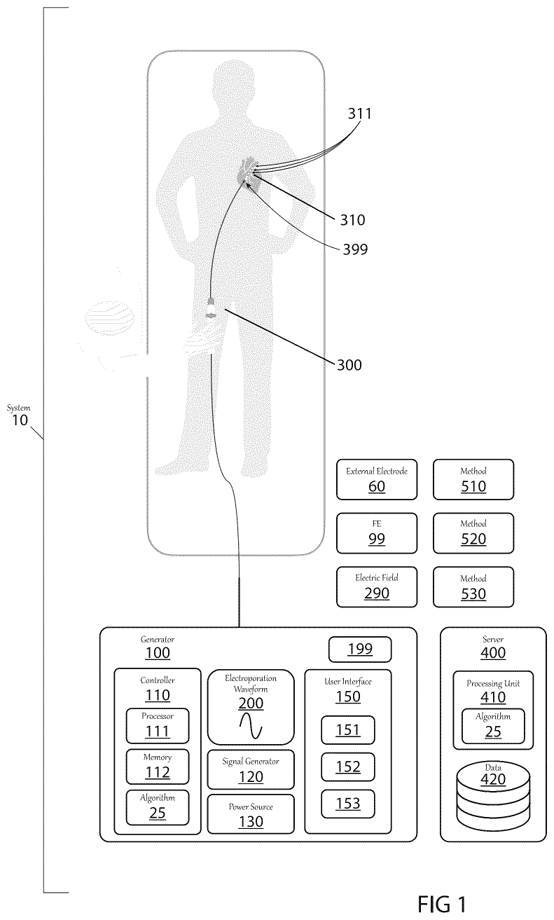

Referring now to FIG. 1, a schematic example of a system comprising a catheter and a generator for providing electroporation is illustrated, consistent with the present inventive concepts. System 10 can include generator 100 comprising a controller 110 configured to control one or more signal generators, such as signal generator 120, each as shown. System 10 can include one or more power supplying assemblies, such as power source 130 of generator 100. Power source 130 can be configured to provide power to signal generator 120. Generator 100 can be configured to deliver electrical energy comprising one or more waveforms, such as electroporation waveform 200, described in detail herein. System 10 can include one or more patient treatment devices, such as catheter 300 shown. Catheter 300 can comprise an array of one or more electrodes, such as electrode array 310 including electrodes 311, as shown. Catheter 300 can operably attach to generator 100 such that electroporation waveform 200 can be provided by generator 100 and delivered to the patient via catheter 300, such as is described herein. Delivery of electroporation waveform 200 to one or more electrodes 311 can result in one or more electric fields, singly or collectively electric field 290, that is generated in tissue proximate electrodes 311 of catheter 300. As described herein, the parameters and method of delivery of electroporation waveform 200 can be configured such that tissue within a portion of field 290 that is sufficient to electroporate the tissue is effectively electroporated, such as is described herein. In some embodiments, one or more components may not form part of generator 100. For example, an external power source can be used and, as such, power source 130 may not comprise part of generator 100. In other examples, the signal generator 120 itself can also comprise power source 130 as opposed to the two components being provided individually. In some examples, controller 110 can be powered by power source 130 while in other examples, controller 110 can be powered by other means.

In some embodiments, electroporation waveform 200 is configured to provide, when delivered to tissue, coherent sine-burst electroporation (CSE). CSE comprises the delivery of high voltage (e.g., at least 100V, or 1500V), phased sine waves to ablate (e.g., irreversibly electroporate) tissue, as described herein.

Generator 100 can include one or more isolation transformers. Sine waves are more compatible with isolation transformers than square waves, because the energy of the sine wave can be concentrated at a single frequency in the passband of the isolation transformer. Isolation transformers are considered the “gold standard” for patient safety because these types of transformers allow the patient's electrical potential to “float” relative to the potential in the generator 100, and all pulsed field ablation (PFA) energy must couple through the transformer's magnetic field to reach the patient. Therefore, any electrical failure on the generator 100 primary side of the transformer does not propagate to the secondary side attached to catheter 300, because of the isolation created by the transformer's magnetic field. By generator 100 providing a sine wave and leveraging the voltage gain made possible by a properly selected isolation transformer, it is possible for generator 100 to generate much higher voltages and thus much higher electric fields in a sine wave-based generator configuration, which will result in greater depth propagation of irreversible PFA.

Additionally, pure sine waves can be easily combined by generator 100 to interfere constructively, by varying the relative phase between multiple sine waves applied to adjacent activated electrodes, as described herein. If an external electrode 60 is connected, and the sine waves of a given frequency applied to adjacent activated electrodes 311 (e.g., electrodes positioned on the endocardial surface) have no phase shift, then a unipolar field is generated between the electrodes 311 and external electrode 60 (e.g., comprising one or more patch electrodes). If the sine waves applied to adjacent activated electrodes 311 are at the same frequency, but 180° out of phase, then the two waves constructively interfere and combine to create a bipolar sine wave of twice the amplitude of that applied to each individual electrode 311. No external electrode 60 (e.g. no return patch electrodes) is needed in this configuration.

System 10 can use unipolar fields to create a deeper lesion for a given peak voltage than a bipolar lesion. This increased depth is due to the field being directed from the endocardial tissue surface contacted by electrodes 311 outwards through the thickness of the heart through the rest of the body (e.g., toward one or more external electrodes 60), but tends to result in a greater degree of neuromuscular stimulation due to the larger number of muscle groups that are located between the electrodes 311 and external electrode 60. Depending on electrode 311 spacing and other factors, unipolar lesions can also result in less uniform ‘fill’ between adjacent electrodes 311, and can include gaps. Bipolar fields maintain their fields locally because the endocardially-positioned electrodes 311 act as both a source and sink, resulting in negligible neuromuscular stimulation and a higher uniformity of fill without gaps between electrodes. Although the more localized nature of a bipolar field leads to less tissue penetration for a given peak voltage, the voltage doubling by driving out of phase can compensate and thus bipolar fields are often preferred in applications like Atrial Fibrillation (AF) where the tissue to be ablated is frequently less than 5 mm in thickness to the epicardial surface.

Finally, another advantage of sine wave based systems is that sine wave based PFA is more efficient than either biphasic or monophasic square wave based PFA. Unlike standard RF ablation which relies on the root-mean-square (RMS) of the AC current to cause resistive heating, electroporation is a field effect which relies on the peak amplitude of the field being generated. For any given amplitude, sine waves have less power, hence less heat generated, than square waves of the same amplitude. Further, a portion of the spectral energy in a square wave which causes the heat is contained within the odd harmonics. These harmonics are at multiples of the fundamental frequency where electroporation is less effective due to the low pass nature of biological tissue. In short, to generate a field of equivalent amplitude to a sine-wave based system, square wave systems require more heat generating energy at frequencies where it will have minimal impact on lesion generation.

System 10 can be configured to provide electroporation at a treatment site of a patient via catheter 300. In particular, the electroporation technique may comprise high-frequency irreversible electroporation, however, in some embodiments, system 10 can be configured to provide a different type of electroporation, such as low frequency non-thermal irreversible electroporation or reversible electroporation, and/or electrolytic electroporation, such as electrolytic electroporation comprising a combination of low and high frequency electroporation.

In some embodiments, when delivering electroporation waveform 200, energy can be delivered between two or more adjacent and/or other endocardially-positioned electrodes, such as electrodes 311 of catheter 300, in a bipolar arrangement, as described herein. Additionally or alternatively, energy can be delivered between an endocardially-positioned electrode 311, and one or more external patient return patches, such as external electrode 60 shown. Delivery of energy between an endocardially-positioned electrode (e.g., electrode 311) and one or more external patch electrodes (e.g., external electrode 60) can be described as delivering energy in a unipolar arrangement. In some embodiments, for example when system 10 utilizes pure sine waves, electroporation waveform 200 can be delivered by generator 100 in a phased-combination arrangement (i.e., a combination of bipolar and unipolar delivery), where these pure sine waves can be combined to interfere constructively, by varying the relative phase between sine waves applied to adjacent activated electrodes (e.g., electrodes 311), and a reference voltage can be provided by generator 100 to external electrode 60 (e.g., one or more patch electrodes positioned on the skin of the patient), such as is described herein.

Unipolar energy delivery methods can be used to create a deeper lesion for a given peak voltage than for a lesion created using a bipolar method utilizing two or more endocardially-positioned electrodes (e.g., electrodes 311), because the direction of the resultant field (e.g., field 290) can be oriented from the contacted endocardial tissue outwards through the thickness of the heart wall tissue and through the rest of the body toward the one or more patch electrodes on the patient's skin. This unipolar energy delivery tends to result in a greater degree of neuromuscular stimulation due to the larger number of muscle groups that are located on the path to the associated patient return electrode (e.g., external electrode 60). In some embodiments, for example depending on catheter 300 and electrode 311 spacing as well as other factors, lesions created using a unipolar energy delivery can result in less uniform ‘fill’ between adjacent electrodes 311, and can possibly include gaps. Bipolar fields maintain their fields locally because the endocardially-positioned electrodes 311 act as both a source and sink, resulting in negligible neuromuscular stimulation and a higher uniformity of fill, without any gaps between electrodes.

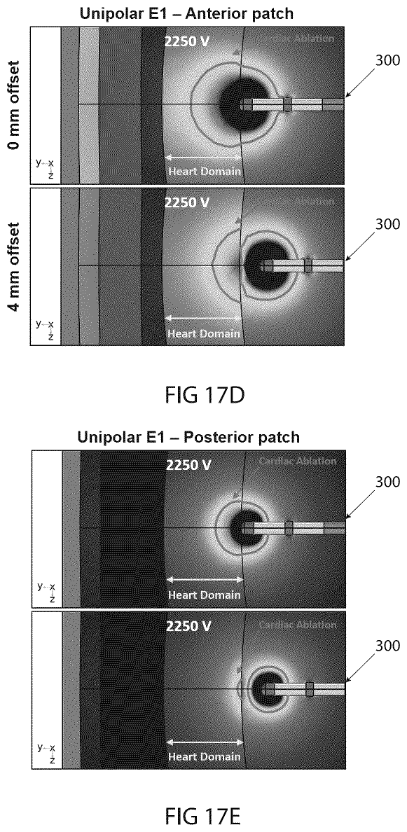

In some embodiments, the placement and/or selection of one or more external patch electrodes (e.g., external electrode 60) can enhance and/or reduce the size of the unipolar component created, by pulling the field in a given direction toward the one or more patch electrodes that are activated. For example, if external electrode 60 comprises a patch electrode that is placed on the patient's skin such that the substrate to be ablated is tissue located between the endocardially-positioned electrodes 311 and the external electrode 60, the size of the created lesion will be enhanced (e.g., deeper depth is achieved and a transmural lesion is created). System 10 can be configured to ablate one or more locations in any chamber of the heart, such as when configured to allow a clinician to ablate any location in the left atrium and/or other heart chamber. In some embodiments, electrode 60 comprises multiple patch electrodes that are positioned on the patient's skin in various locations, with each electrode independently activatable (e.g., configured to be selected as a return electrode), such as to activate particular patch electrodes to direct the field in one or more of anterior, posterior, superior and/or inferior directions, such as to cause a transmural lesion to be created in the cardiac wall at the location of the associated electrode 311. Unipolar energy delivery using an external electrode 60 comprising multiple patch electrodes can be similar to unipolar energy delivery and/or phased-combination energy delivery described herebelow in reference to FIG. 17.

Controller 110 can comprise a module (e.g., an electronics module) that can be configured to perform and/or facilitate one or more functions of system 10, such as one or more processes; energy deliveries, such as delivery of an electroporation waveform; data analyses; data transfers; signal processing; and/or other functions of system 10 (“functions of system 10” or “system functions” herein). Controller 110 can comprise one or more electronic elements, electronic assemblies, and/or other electronic components, such as components selected from the group consisting of: microprocessors; microcontrollers; state machines; memory storage components; analog-to-digital converters; rectification circuitry; filters and other signal conditioners; sensor interface circuitry; transducer interface circuitry; and combinations of one, two, or more of these. For example, controller 110 can include at least one processor and at least one memory storage component, such as processor 111 and memory 112, each shown. Memory 112 can be coupled to processor 111, and memory 112 can store instructions used by processor 111 to perform one or more algorithms of system 10. For example, system 10 can comprise one or more algorithms, algorithm 25 shown, that are performed by processor 111 and/or another similar arrangement of a processor and instructions stored in memory. Algorithm 25 can comprise one or more machine learning, neural net, and/or other artificial intelligence algorithms (“AI algorithm” herein). All or a portion of algorithm 25 can be integrated into (e.g., stored in the memory of) one, two, or more of the various components of system 10, such as a device or other component of system 10 comprising a processor and/or a console of system 10. Controller 110 can comprise a microprocessor or other processing unit which enables it to receive input data and provide output signals based on the input data. In some embodiments, controller 110 is configured to receive inputs from a user input device and/or from an automated computing device. Inputs from a user input device may come directly to controller 110 and/or may be provided via one or more other electronic devices. The inputs received by controller 110 can relate to particular parameters that define an electroporation waveform. For example, the received parameters can comprise a desired frequency, intensity, duration, phase, cycle length or other parameter of the waveform. As an output, controller 110 can be configured to provide signaling which is configured to interact with signal generator 120 where the signaling is configured to cause signal generator 120 to generate a desired electroporation waveform.

In some embodiments, generator 100 and/or another component of system 10 can include a user interface, such as user interface 150 of generator 100 shown, such as a user interface configured to provide and/or receive information to and/or from an operator of system 10. User interface 150 can be integrated into generator 100 as shown. Alternatively or additionally, user interface 150 can comprise a component separate from generator 100, such as a display separate from, but operably attached to, generator 100. User interface 150 can include one, two, or more user input and/or user output components. For example, user interface 150 can comprise a joystick, keyboard, mouse, touchscreen, speaker, light, transducer, and/or another human interface device, user interface device 151 shown. In some embodiments, user interface 150 comprises a display (e.g., a touchscreen display), such as display 152, also shown. In some embodiments, processor 111 can provide a graphical user interface, GUI 153, to be presented on and/or provided by display 152. Algorithm 25 can be configured to perform one or more software routines that enable user control of one or more functions of system 10. The one or more software routines performed by algorithm 25 can comprise a graphical user interface, such as GUI 153. User interface device 151 can include an input and/or output device selected from the group consisting of: a speaker; an indicator light, such as an LED indicator; a haptic feedback device such as a device comprising a vibrational alert component; a foot pedal; a switch, such as a momentary switch; a microphone; a camera, for example when processor 111 enables eye tracking and/or other input via image processing; and combinations of these. In some embodiments, catheter 300 includes at least a portion of user interface 150, such as a user input device 151, for example when functional element 399 of catheter 300 comprises a button or other interface device 151 of user interface 150. Additionally or alternatively, catheter 300 can include a user interface device 151 including a user output device, such as a light or a speaker, for example a light configured to indicate a readiness condition of system 10 (e.g., a light configured to indicate when system 10 is and/or is not ready to provide electroporation waveform 200).

In some embodiments, system 10 includes a data storage and processing device, server 400. Server 400 can comprise an “off-site” server (e.g., outside of the clinical site in which patient image data is recorded), such as a server owned, maintained, and/or otherwise provided by the manufacturer of system 10. Alternatively or additionally, server 400 can comprise a cloud-based server. Server 400 can include processing unit 410 shown, which can be configured to perform one or more functions of system 10, such as one or more functions described herein. Processing unit 410 can include one or more algorithms, such as algorithm 25 described herein. Processing unit 410 can comprise a memory (not shown), which can store instructions for performing algorithm 25. Server 400 can be configured to receive and store various forms of data, such as: treatment data, diagnostic data, planning data, and/or procedural outcome data collected by system 10, data 420. In some embodiments, data 420 can comprise data collected from multiple patients (e.g., multiple patients treated with system 10), such as data collected during and/or after clinical procedures where electroporation waveform 200 was delivered to the patient via system 10. In some embodiments, generator 100 and server 400 can communicate over a network, for example, a wide area network such as the Internet. Alternatively or additionally, system 10 can include a virtual private network (VPN) through which various devices of system 10 transfer data.

As described herein, the one or more functions of system 10 performed by controller 110 and/or processing unit 410 can be performed by either or both devices. For example, in some embodiments, treatment data can be collected by controller 110 of generator 100. The treatment data can then be transferred to server 400, where the data is processed, for example to identify one or more trends, such as one or more trends in the effectiveness of various parameters of electroporation waveform 200 described herein. The insight attained from data processing of server 400 can then be transferred back to generator 100, for example to inform a decision-making process (e.g., a decision made by algorithm 25 and/or an operator of system 10) regarding one or more parameters of electroporation waveform 200 to be provided to treat a patient.

In some embodiments, algorithm 25 is configured to adjust (e.g., automatically and/or semi-automatically adjust, such as an adjustment performed based on one or more biases included in algorithm 25, as described herein) one or more operational parameters of system 10, such as one or more of the parameters of electroporation waveform 200 described herein. In some embodiments, algorithm 25 is configured to adjust an operational parameter based on one or more sensor signals, such as a sensor signal provided by a sensor-based functional element of the present inventive concepts as described herein. Algorithm 25 can be configured to adjust (e.g., automatically adjust and/or recommend the adjustment of) an operational parameter selected from the group consisting of: to which one or more electrodes of a set of electrodes to provide electroporation waveform 200 (e.g., one or more electrodes 311 and/or one or more external electrodes 60); with which energy modality to deliver energy; the phase angle between two or more signals of electroporation waveform 200; to which tissue locations to deliver energy, such as locations determined by analyzing cardiac mapping data; to which external electrode 60 to provide electroporation waveform 200 such as to direct the generated electric field toward target tissue; and combinations of two or more of these.

In some embodiments, algorithm 25 is configured to determine one or more parameters of a stimulation waveform. In these embodiments, algorithm 25 can comprise one or more biases, such as a bias to create a stimulation waveform that tends toward: a particular frequency range; a particular ratio of bipolar-to-unipolar energy delivery; a particular phase difference between included sine waves; a particular voltage or range of voltages; a particular delay between energy deliveries such as a particular inter-pulse delay; and combinations of one or more of these.

It will be appreciated that the term “signaling” used herein refers to one or more signals provided by controller 110 that comprise information for interpretation by signal generator 120 or, optionally, by another component of system 10. Signaling can be provided by way of one or more wired connections and/or wireless modes of data communication. As mentioned above, the signaling can be provided to signal generator 120 as per the embodiment of FIG. 1, and/or it may be provided to a power source itself, the power source being configured to generate an electroporation waveform, as described herein.

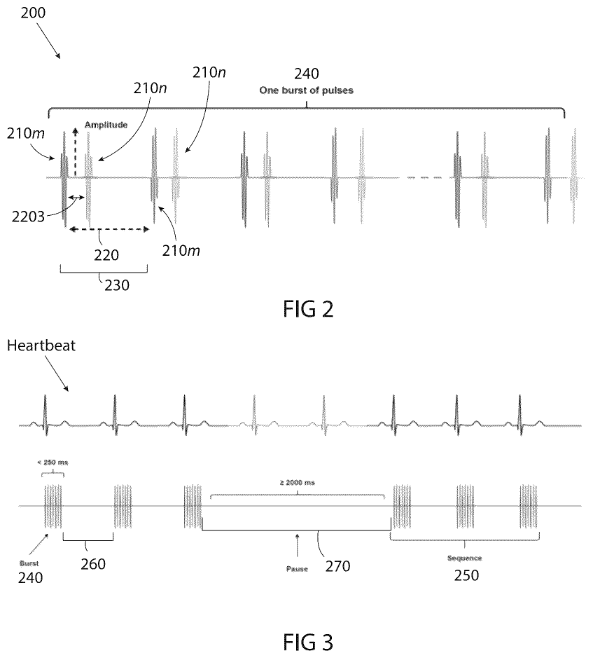

In some embodiments, algorithm 25 can be configured to cause system 10 to perform a method, method 510, the method 510 providing an electroporation waveform (e.g., electroporation waveform 200 described herein) to an ablation device, such as catheter 300. Method 510 can comprise providing signaling from controller 110 to a signal generator 120. The signaling can be configured to cause the signal generator 120 to produce an electroporation waveform 200, and the electroporation waveform can comprise a plurality of pulses, where each energy pulse is separated by an inter-pulse delay period, such as energy pulses 210 separated by inter-pulse delay periods 220 as described in reference to FIG. 2 and otherwise herein. In some embodiments, each energy pulse can comprise one or more distinct sinewave signals, as described herein. Method 510 can further comprise generating the electroporation waveform based on the signaling (e.g., signal generator 120 can generate electroporation waveform 200 based on the signaling from controller 110). Method 510 can include providing the electroporation waveform 200 from the signal generator to an electroporation catheter, such as catheter 300, for the provision of the electroporation waveform 200 to a target area.

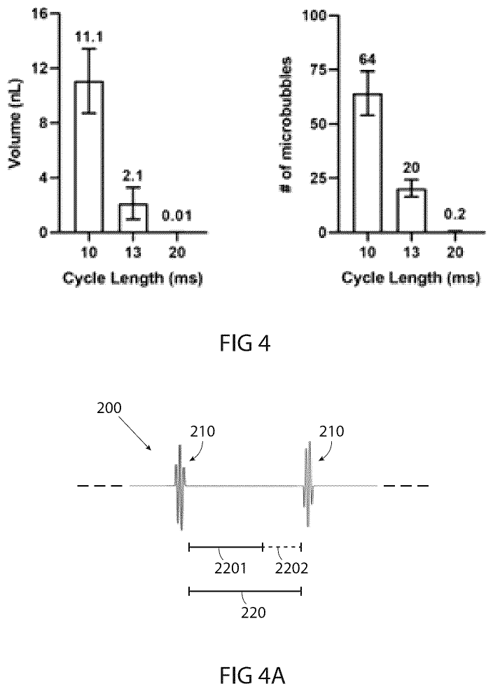

In some embodiments, algorithm 25 can be configured to cause system 10 to perform a method, method 520, the method 520 providing an electroporation waveform and one or more compensation signals, such as is described herebelow in reference to FIGS. 5-7 and otherwise herein. In some embodiments, method 520 comprises providing signaling (e.g., signaling from controller 110) to cause the provision of an electroporation waveform 200, where the electroporation waveform 200 comprises one or more sequentially provided energy pulses 210 where each energy pulse 210 is configured to cause electroporation. Method 520 can further comprise providing signaling from controller 110 to cause the signal generator 120 to provide one or more compensation signals (e.g., compensation signal 2102 described herein) where the one or more compensation signals are configured to reduce a build-up of charge at a treatment location of a patient, the built-up charge caused by one or more of the plurality of stimulation signals (e.g., the plurality of energy pulses 210). In some embodiments, the controller 110 may be configured to provide this signaling to a signal generator 120 to cause the signal generator 120 to provide the electroporation waveform 200 comprising the energy pulses and the charge-reducing compensation signals. In some embodiments, the compensation signals are added to the electroporation waveform 200.

In some embodiments, algorithm 25 can be configured to cause system 10 to perform a method, method 530. Method 530 can comprise providing first signaling configured to cause the application of a first potential difference between a first electrode and a second electrode (e.g., a first electrode 311 and a second electrode 311 of catheter 300) where the second electrode is a non-neighboring (e.g., non-adjacent) electrode to the first electrode. Method 530 can further comprise providing second signaling configured to cause the application of a second potential difference between a third electrode and a fourth electrode (e.g., a third electrode 311 and a fourth electrode 311 of catheter 300) where the third electrode is a neighboring electrode (e.g., an adjacent electrode) to the first electrode and the fourth electrode is a non-neighboring electrode to the third electrode. In some embodiments, the first potential difference is applied between the first electrode and the second electrode asynchronously from the application of the second potential difference between the third electrode and the fourth electrode. Method 530 can be applied throughout a plurality of electrodes and electrode pairs, resulting in a contiguous cellular ablation lesion.

Signal generator 120 can comprise any suitable signal generator for generating the electroporation waveform which comprises an electric signal for energizing one or more of the electrodes 311 of catheter 300. That is, the electroporation waveform is configured to cause the application of voltage electric fields to biological tissue via electrodes 311 of catheter 300. In particular, signal generator 120 can comprise a sinewave generator that is configured to generate signals comprising one or more sinewaves. Power source 130 can comprise any suitable device for providing electrical power to at least signal generator 120.

Catheter 300 can comprise a plurality of electrodes 311 (e.g., electrodes 311 of electrode array 310 described herein), the electrodes 311 being positionable either on or near target tissue that is to be subjected to the electroporation of system 10. The electrodes 311 can be configured such that potential differences are generated between activated electrodes 311 responsive to the electroporation waveforms provided thereto.

In some embodiments, system 10 and/or one or more components of system 10 further comprise one or more functional elements (“functional element” herein) such as functional element 99, functional element 199 of generator 100, and/or functional element 399 of catheter 300, each shown. Each functional element can comprise at least two functional elements. Each functional element can comprise one or more elements selected from the group consisting of: sensor; transducer; and combinations thereof. Each functional element of system 10 can comprise a sensor configured to produce a signal. Each functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; an impedance sensor; a charge sensor; and combinations thereof. Each functional element can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. In some embodiments, system 10 can further comprise one or more algorithms, such as algorithm 25 described herein, configured to process the signal produced by a sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof. In some embodiments, functional element 399 comprises one or more vacuum ports that are fluidly connected (e.g., via a lumen of catheter 300) to a functional element 199 comprising a source of vacuum. In these embodiments, vacuum can be applied to functional element 399 by element 199, such that one or more portions of catheter 300 (e.g., one or more portions including one or more electrodes 311) are maintained in contact with tissue via the applied vacuum.

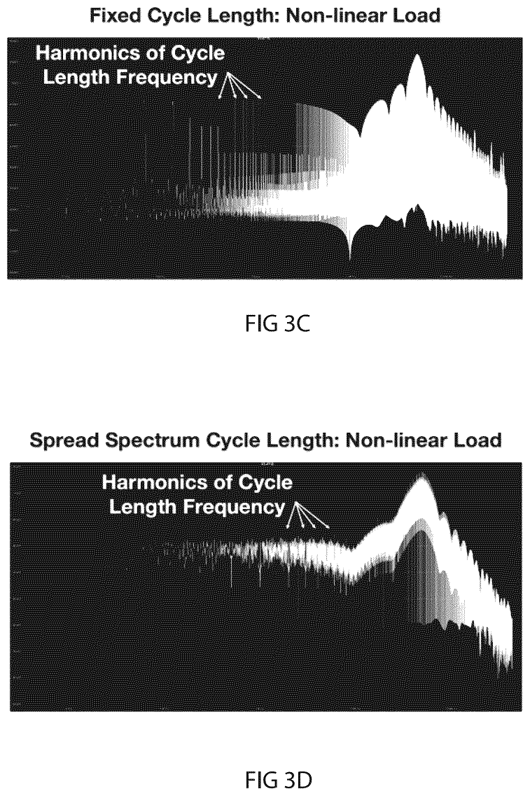

Referring now to FIG. 2, an example of a portion of an electroporation waveform is illustrated, consistent with the present inventive concepts. In some embodiments, the waveform of FIG. 2 comprises a series of energy pulses with a fixed inter-pulse delay, for example as described herein. FIG. 2 shows an example electroporation waveform 200 comprising a plurality of energy pulses 210 and a plurality of inter-pulse delay periods 220, each period 220 arranged between pairs of energy pulses 210 such that the energy pulses 210 are time-spaced energy pulses. A single cycle 230 of the electroporation waveform 200 can be defined from the start of a first energy pulse to the start of the next energy pulse. In some embodiments, electroporation waveform 200 comprises a series of multiple cycles 230, burst 240 shown. It will be appreciated that this is simply an example, as a cycle 230 could equally be defined between any two like-points on consecutive energy pulses 210 or consecutive delay periods 220. In FIG. 2, voltage is provided along the y-axis and time is provided along the x-axis. In some embodiments, inter-pulse delay period 220 comprises two portions, such as a fixed inter-pulse delay period 2201 and a variable inter-pulse delay period 2202, not shown, but such as is described in reference to FIG. 4A and otherwise herein.

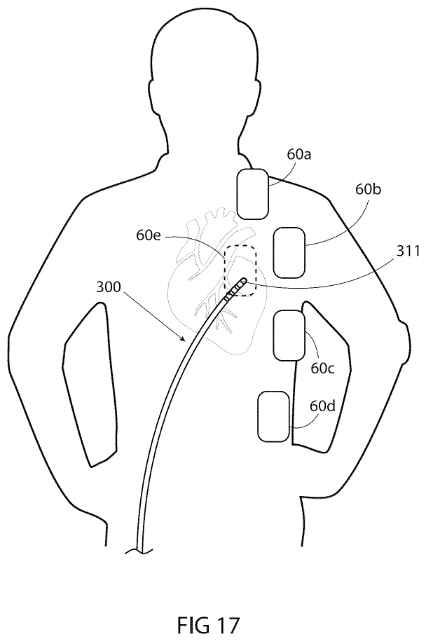

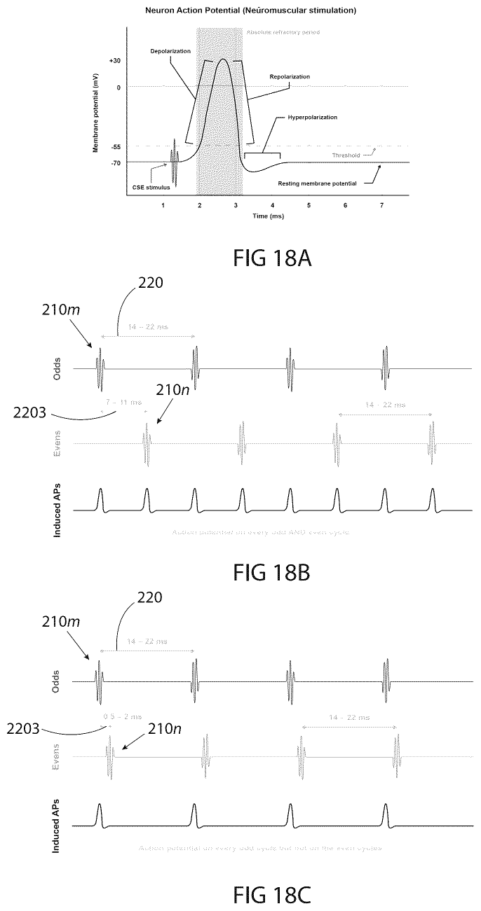

In some embodiments, system 10 is configured to provide a first portion of electroporation waveform 200 from a first set of electrodes 311 and a second portion of an electroporation waveform 200 from a second set of electrodes 311, such as delivery of electroporation waveform 200 in an interleaved pattern, for example as described in reference to FIGS. 18A-C, and otherwise herein. In some embodiments, burst 240 can comprise a first set of energy pulses, energy pulses 210m, and a second set of energy pulses, energy pulses 210n, where energy pulses 210m are delivered from a first set of electrodes 311 (e.g., electrodes 311a, c shown in FIG. 15) and energy pulses 210n are delivered from a second set of electrodes 311 (e.g., electrodes 311b, d shown in FIG. 15). In some embodiments, each cycle 230 can comprise an energy pulse 210m and an energy pulse 210n. Electrode 311b can be located between electrodes 311a, c, as described herebelow, such that electroporation waveform 200 is provided in an interleaved pattern from non-neighboring electrodes. In some embodiments, inter-pulse delay period 220 comprises the duration between a first set of energy pulses 210 that are delivered from a first set of electrodes 311 (e.g., energy pulses 210m), where, in some embodiments, at least a second energy pulse 210 (e.g., energy pulses 210n) is delivered from a second set of electrodes 311 within the inter-burst delay period 220 of the first set of energy pulses 210m. Inter-pulse delay period 220 can include interleaving offset period 2203, where interleaving offset period 2203 comprises the time delay between the end of the first energy pulse 210m and the start of the second energy pulse 210n. In some embodiments, interleaving offset period 2203 comprises a duration equal to one half of inter-pulse delay period 220, such that each energy pulse 210 (e.g., energy pulses 210m and 210n) are delivered with an equal frequency, for example as shown in FIG. 18B. Alternatively or additionally, interleaving offset period 2203 can comprise a duration that is less than half the duration of inter-pulse delay period 220, for example such that energy pulses 210m and 210n are delivered within the first half of cycle 230, such as is shown in FIG. 18C.

In some embodiments, system 10 is configured to provide portions of electroporation waveform 200 to two, three, four, or more sets of electrodes 311 in an interleaved pattern. For example, a first portion of electroporation waveform 200, energy pulses 210m, can be delivered via every third electrode 311 (e.g., electrodes 311 of a linear array 310 of sequentially numbered electrodes 1-12), such as electrodes 311-1,4,7, and 10. A second portion, energy pulses 210n, can be delivered from electrodes 311-2,5,8, and 11, and a third portion, energy pulses 2100, can be delivered from electrodes 311-3,6,9, and 12. Cycle 230 can comprise the delivery of one of each energy pulse 210m, n, o. In some embodiments, interleaving offset period 2203 between energy pulses 210m, n and 210n, o can comprise similar and/or dissimilar durations of time. In some embodiments, the total offset period between a first energy pulse (e.g., energy pulse 210m) and a last energy pulse (e.g., energy pulse 2100) of a cycle 230 of energy pulses can comprise a period of less than the refractory period of action potentials (APs) induced by the first energy pulse 210m, for example as described in reference to FIG. 18C and otherwise herein.

As provided for herein, the electroporation waveform 200 comprises a total waveform that is provided for the application of reversible electroporation, irreversible electroporation, or both. In one or more embodiments, the electroporation waveform 200 can specifically be an ablation waveform which comprises ablation pulses configured to cause cell death via irreversible electroporation.

The electroporation waveform 200 can comprise a plurality of energy pulses 210. In some embodiments, each energy pulse 210 comprises one or more distinct sinewaves signals. For example, electroporation waveform 200 can comprise a plurality of energy pulses 210 where each energy pulse 210 comprises a plurality of distinct sinewave signals. It will be appreciated that herein the phrase “distinct sinewave signals” is used in order to define multiple sinewave signals which are distinguishable from one-another. This configuration of signals may relate to sinewaves which are entirely discrete from one-another in that they start at a reference voltage, cross through the reference voltage and end at substantially the same reference voltage prior to the next sinewave beginning. The reference voltage can be zero Volts, but it is also possible for the reference voltage to have a different value. Distinct sinewaves can also relate to sinewaves which are partially overlapping but in such a manner that the sinusoidal waveform of each sinewave is distinguishable. Sinewaves which overlap in such a way that they constructively interfere to produce sinewaves of a summed greater amplitude, or sinewaves which form a sinewave or other waveform with a broader linewidth than any of the original sinewaves (e.g., where the constituent sinewaves are not separately distinguishable), would not necessarily be considered distinct sinewave signals. In some embodiments, each energy pulse 210 can comprise a single distinct sinewave. It will be appreciated that “a single distinct sinewave” defines that only one distinct sinewave is provided absent of any other distinguishable sinewaves in each energy pulse 210. This configuration does not prohibit multiple sinewaves being superimposed upon one-another if the superposition of sinewaves provides an effective single distinct sinewave, as defined above. In examples where a single distinct sinewave is used in each energy pulse 210, the frequency of the single distinct sinewave can be between 20 kHz and 200 kHz and the amplitude can be between 500V and 3 kV, which would provide a maximum to minimum amplitude difference of 1 kV to 6 kV. The frequency can particularly be approximately 50 kHz. It will be appreciated that sine waves can be applied to two electrodes (e.g., two neighboring or non-neighboring electrodes 311) and/or two groups of one or more electrodes, each in order to provide for a potential difference therebetween. In the case of a phase difference of 0 degrees between a sine wave applied to a first electrode (or first group of electrodes) and a sine wave applied to a second electrode (or second group of electrodes), the resultant signal will be unipolar, for example as described herein. In the case of a phase difference of 180 degrees between a sine wave applied to a first electrode and a sine wave applied to a second electrode, the resultant signal will be bipolar (e.g., when no signal is applied to an external electrode 60), for example as described herein. Alternatively, the phase difference may have another value which will result in a signal having both unipolar and bipolar components, for example when configured as a phased-combination energy delivery as is described herein.

Electroporation waveform 200 can comprise a pulsed waveform, and the signal between the initiation of a first pulse and initiation of a subsequent pulse can be defined as a single cycle 230 of the electroporation waveform 200. The provision of the energy pulses 210 to electrodes 311 of catheter 300 provides for the generation of potential differences which in turn create the electric fields which cause the desired electroporation. It will be appreciated that causing electroporation may refer to causing either reversible or irreversible electroporation. Between a first energy pulse and a subsequent energy pulse there can be an inter-pulse delay period 220 during which no energy pulses 210 are provided. It will be appreciated that there may be some noise or other signals provided during the inter-pulse delay period 220, but no pulses having pulse characteristics suitable for stimulating electroporation are provided during the delay period 220. The provision of non-electroporation-causing pulses during the inter-pulse delay period 220 may allow, for example, for the provision of offset pulses that are provided in order to counteract charge build-up in a patient resultant from foregoing pulses. By providing for an inter-pulse delay period 220 during which no electroporation-causing pulses are provided, a reduction of microbubbles can be achieved as compared to systems which do not provide for such an inter-pulse delay period 220. In some embodiments, inter-pulse delay period 220 is of sufficient duration such that the electroporation waveform 200 comprises a series of energy pulses 210, such that the electroporation waveform 200 provides pulsed ablation energy in contrast to a continuous wave ablation energy. For example, the inter-pulse delay period 220 can be of at least 10 ms, such as up to 25 ms. For example, the percentage of a single cycle 230 over which the energy pulse 210 is provided can be 5%, 4%, 3%, 2%, 1% or a different percentage of the cycle 230 (e.g., but not greater than 5%). Most particularly, the percentage of a single cycle 230 over which the energy pulse 210 is provided can be at least 0.1%, at most 0.3%, and/or it can be nominally 0.2%.

In summary, a single cycle 230 of the electroporation waveform 200 can comprise an energy pulse 210 (e.g., an energy pulse comprising one or more distinct sinewaves) followed by an inter-pulse delay period 220 during which no electroporation-causing energy pulses 210 are provided.

Further, the amplitude and frequency of each energy pulse 210 can be sufficient to cause each energy pulse to provide for ablation of a target tissue. In such cases, the energy pulses 210 may be referred to as ablation pulses 210. For example, the frequency of the pulses can be at least a minimum frequency X kHz and/or no more than a maximum frequency Y kHz; and the amplitude can be between at least a minimum voltage of M Volts, and/or nor more than a maximum voltage of N Volts. By way of example only, the inter-pulse delay period can be between a minimum of 2 ms, and/or a maximum of 2000 ms. In other embodiments, the inter-pulse delay period 220 can be at least 15 ms, and/or no more than 100 ms, such as when the period 220 is at least 15 ms and/or no more than 50 ms.

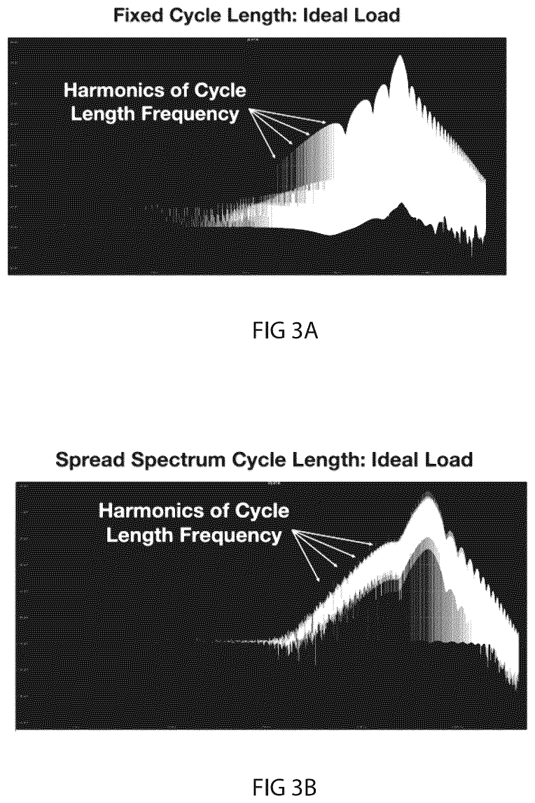

Referring additionally to FIG. 3, an example of a portion of an electroporation waveform is illustrated, consistent with the present inventive concepts. Electroporation waveform 200 of FIG. 3 can comprise multiple bursts 240, such as burst 240 shown in FIG. 2. In some embodiments, electroporation waveform 200 of FIG. 3 comprises one or more time-spaced groupings of bursts 240, sequences 250 shown. Each sequence 250 can include a series of one, two, or more bursts 240, where each burst 240 is separated by an inter-burst delays period 260. In some embodiments, electroporation waveform 200 can include two or more sequences 250, such as two or more sequences 250 each separated by an inter-sequence delay period 270.

As described herein, a burst 240 can comprise one or more energy pulses 210 each separated by an inter-pulse delay period 220. Inter-pulse delay period 220 can include a fixed delay period and/or a variable delay period, such as both a fixed period and a variable period (e.g., a fixed and variable period that together define the duration of inter-pulse delay period 220) such as fixed inter-pulse delay period 2201 and variable inter-pulse delay period 2202 described herein. A sequence 250 can comprise one or more bursts 240 each separated by an inter-burst delay period 260. Inter-burst delay period 260 can include a fixed delay period and/or a variable delay period (e.g., a fixed and variable period that together define the duration of inter-burst delay period 260). An electroporation waveform 200 (e.g., a waveform 200 that is provided to tissue in total in a single energy delivery process) can comprise one or more sequences 250 each separated by an inter-sequence delay period 270. Inter-sequence delay period 270 can include a fixed delay period and/or a variable delay period (e.g., a fixed and variable period that together define the duration inter-sequence delay period 270).

Each energy pulse 210 can comprise at least one distinct sine wave. In some embodiments, energy pulse 210 comprises no more than 50 distinct sine waves, such as no more than 10 distinct sine waves. In some embodiments, one or more energy pulses 210 of electroporation waveform 200 (e.g., two consecutive energy pulses 210 of a burst 240) comprise a different number of distinct sine waves. In some embodiments, the number of sine waves of energy pulse 210 is variable, such as a quantity of sine waves that is generated as a pseudo-random (e.g., a pseudo-random number generated by algorithm 25), and/or when the number of sine waves of an energy pulse 210 is determined by system 10 (e.g., determined by algorithm 25 based on one or more system and/or patient parameters, such as based on the temperature of tissue proximate electrodes 311). For example, in some embodiments, a functional element (e.g., functional element 99), such as a functional element comprising a thermocouple, is used to monitor the temperature of one or more electrodes 311 of catheter 300. Energy pulses 210 can be delivered at a rate of at least 1 energy pulse per cardiac cycle, and/or no more than 100 energy pulses per cardiac cycle, for example at a rate of approximately 50 energy pulses per cardiac cycle. In some embodiments, until the recorded temperature exceeds a threshold, such as a threshold of at least 38° C. or 45° C., the delivery rate can remain constant. Once the recorded temperature exceeds a threshold, algorithm 25 can be configured to lower the rate of pulses per cardiac cycle (e.g., lower the rate proportionally based on the recorded temperature). In some embodiments, if the recorded temperature falls below the threshold, the rate can be increased (e.g., increased to the original rate). In some embodiments, the threshold temperature can comprise a temperature of at least 38° C., and/or no more than 80° C.

Inter-pulse delay period 220 can comprise a duration of at least 1 ms, such as at least 15 ms. In some embodiments, inter-pulse delay period 220 comprises a duration of no more than 2000 ms, such as no more than 60 ms.

Interleaving offset period 2203 can comprise a duration of at least 0.5 ms. In some embodiments, interleaving offset period 2203 comprises a duration of no more than 2 ms.

Inter-burst delay period 260 can comprise a duration of at least 100 ms. In some embodiments, inter-burst delay period 260 comprises a duration of no more 5000 ms.

Inter-sequence delay period 270 can comprise a duration of at least 2000 ms, such as at least 5000 ms. In some embodiments, inter-sequence delay period 270 comprises a duration of no more than 20000 ms, such as no more than 10000 ms.

Controller 110 can be further configured to set the inter-pulse delay period 220 based on received conductivity measurement data. Selection of the inter-pulse delay period 220 may be achieved by way of acquiring the inter-pulse delay period 220 from a look-up table based on the conductivity measurement. Such a look-up table can be stored in memory as part of controller 110 (e.g., stored in memory 112) or it can be stored in memory separate from controller 110 with which controller 110 is in communication. Alternatively, any other suitable method for obtaining the inter-pulse delay period 220 based on the received conductivity measurement can be used. Providing for the selection of the inter-pulse delay period 220 based on the received conductivity measurement can allow for the inter-pulse delay period 220 to be tuned to a target tissue to be ablated, where the conductivity measurement is a conductivity measurement of the target tissue. This configuration may be beneficial, as the conductivity of different types of tissues can impact microbubble formation and, as such, the inter-pulse delay period 220 may need to be increased in order to avoid or reduce microbubble formation. Alternatively, certain tissue types may be less prone to microbubble formation and, as such, the inter-pulse delay period 220 may be able to be reduced without significantly increasing microbubble formation.

The received conductivity measurement data can be received in response to controller 110 providing signaling to a conductivity sensor (e.g., a functional element 99 and/or 399 comprising one or more conductivity sensors) configured to cause the conductivity sensor to take a conductivity measurement. Controller 110 can further be configured to receive the conductivity measurement data from the conductivity sensor. Alternatively, the conductivity measurement data can be received from a user input device. Thus, controller 110 can be configured to receive conductivity data either by way of controlling an external conductivity sensor or by way of user input. This may provide for variable functionality for a user, depending on the situation or the availability of a coupled conductivity sensor or it may provide a back-up redundancy in the case that a conductivity sensor is not functioning properly.

Referring additionally to FIGS. 3A-D, graphs of the harmonics of various frequencies under varying conditions are illustrated, consistent with the present inventive concepts. FIG. 3A shows an example of the harmonics of the frequency of a fixed cycle length when delivered to an ideal resistive load. FIG. 3B shows a representative example of the reduction of the harmonics of the frequency of a fixed cycle length when delivered to an ideal resistive load, using 20 cycles with an even step distribution to spread the spectrum. Reduction is approximately 12 dB at each harmonic. FIG. 3C shows an example of the harmonics of the frequency of a fixed cycle length when delivered to a representative non-linear load. FIG. 3D shows a representative example of the reduction of the harmonics of the frequency of a fixed cycle length when delivered to a representative non-linear load using 20 cycles with an even step distribution to spread the spectrum. Reduction is approximately 12 dB at each harmonic.

In some embodiments, energy pulses 210 that are separated by an inter-pulse delay period 220 produce energy at a frequency that is the inverse of the inter-pulse period frequency. For example, an inter-pulse period of 20 ms would produce a frequency of 50 Hz with its even harmonics. The PFA energy pulse in combination with this lower pulse repetition frequency is functionally an Amplitude Modulated (AM) signal where the PFA energy pulses are square wave AM modulated by the pulse repetition frequency. Most tissue exhibits a partial or total non-linear response to this form of energy delivery, and in the same way that a non-linear circuit element like a diode can rectify and demodulate an AM radio wave, if the pulse repetition frequency is low enough, periodic enough, and of sufficient amplitude, muscle stimulation is possible. By varying the period using a variable inter-pulse delay, the resultant low frequency spectrum can be spread, thus reducing the amplitude at any given frequency (e.g., reducing the likelihood of muscle stimulation). This reduction in harmonics is illustrated in FIGS. 3A-D.

In some embodiments, system 10 is configured to create lesions of ablated tissue by delivering electroporation waveform 200 to tissue via electrodes 311 of catheter 300 and/or external electrode 60 (e.g., one or more patch electrodes positioned on the patient's skin). For example, system 10 can deliver unipolar energy pulses 210 between one or more electrodes 311 and one or more external electrodes 60, as described herein. The unipolar signal can comprise a voltage of at least 1000V, such as at least 1500V and/or no more than 4000V, such as no more than 3000V. The unipolar signal can comprise a frequency of at least 10 kHz, such as at least 25 kHz, and/or a frequency of no more than 100 kHz, such as no more than 75 kHz. In some embodiments, a burst (e.g., burst 240) of unipolar energy pulses 210 can comprise approximately 3 or 4 energy pulses 210 that are collectively delivered within no more than 500 ms, such as within 250 ms. Unipolar energy deliveries can produce lesions of ablated tissue with a lesion depth of at least 5 mm, such as at least 10 mm, 15 mm, or 20 mm.

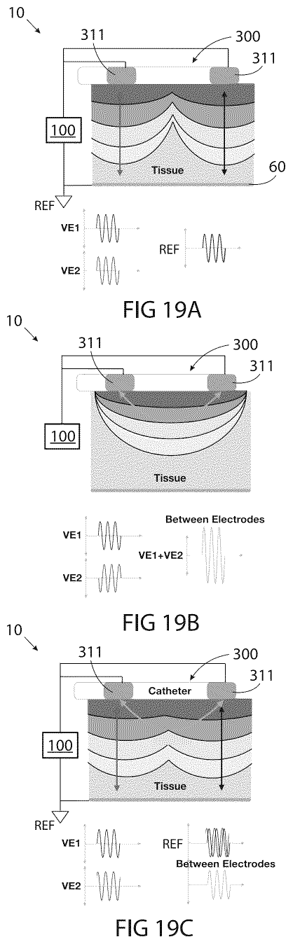

In some embodiments, system 10 is configured to deliver energy pulses 210 where the energy pulses comprise a voltage of at least 1500V, such as at least 2000V, and/or no more than 4000V, such as no more than 3500V. As described herein, in some embodiments, energy pulse 210 can comprise at least a first signal that is provided to at least a first electrode and at least a second signal that is provided to at least a second electrode. As used herein, unipolar energy delivery can comprise energy delivery where the first signal is provided to a first internal electrode (e.g., an endocardially-positioned electrode), such as electrode 311 positioned proximate target tissue, and the second signal is provided to a patch electrode, such as external electrode 60 (e.g., external electrode 60 positioned away from the target tissue, such as on the skin of the patient). Bipolar energy delivery can comprise energy delivery where the first signal is provided to a first internal electrode (e.g., an endocardially-positioned electrode), such as a first electrode 311 positioned proximate target tissue, and the second signal is provided to a second internal electrode (e.g., an endocardially-positioned electrode), such as a second electrode 311 positioned proximate the first electrode 311 and the target tissue. When energy is delivered in a bipolar manner, no signal is provided to a patient patch comprising an electrode, such as external electrode 60, such that the only electric potential that is created by the delivery of the electric signals is generated between the first and second electrodes 311. Phased-combination energy delivery can comprise energy delivery where the first signal is provided to a first internal electrode 311, and the second signal is provided to a second internal electrode 311, and can comprise a third signal, where the third signal comprises a reference signal that is provided to one or more patch electrodes, such as external electrode 60. When energy is delivered in a phased-combination energy delivery manner, a primary electric field is generated between the first and second electrodes 311, and secondary electric fields are generated between each of the first and second electrodes 311 and external electrode 60 (e.g., one or more patch electrodes positioned in one or more locations on the patient's skin). In some embodiments, the first and second signals comprise sine waves, where the phase angle between the sine waves can be between 0° and 180°. The phase angle between the first and second signals can be adjusted to vary the relative strength of the primary electric field generated between the first and second electrodes 311 and the secondary electric fields generated between each of the first and second electrodes 311 and external electrode 60. For example, a phase angle of 180° maximizes the primary electric field relative to the secondary electric fields. As the phase angle decreases (approaches 0°), the field strength of the primary field decreases, as the potential difference between the first and second electrodes 311 decreases. Unipolar, bipolar, and phased-combination energy delivery can be configured as described in reference to FIGS. 19A-C and otherwise herein.

In some embodiments, energy pulses 210 of electroporation waveform 200 comprise sine waves, for example as described herein. In some embodiments, the cycle length of the sine wave is at least 1 ms, such as at least 10 ms, and/or no more than 200 ms, such as no more than 50 ms. In some embodiments, the number of sine waves per pulse (e.g., per energy pulse 210) is no more than 10, such as 1 or 2 sine waves per pulse. Limiting the number of sine waves per pulse can reduce the likelihood of formation of microbubbles. In some embodiments, the number of cycles (e.g., cycle 230) of electroporation waveform 220 required to generate a maximum achievable lesion in tissue is at least 30 cycles, such as at least 100 cycles, and/or no more than 600 cycles, such as no more than 300 cycles. In some embodiments, bursts 240 of electroporation waveform 200 comprise a length of no more than 250 ms, such that electroporation waveform 200 can be provided in a manner that is synchronized with the ventricular refractory period to reduce ventricular excitation.

Referring now to FIGS. 4 and 4A, graphs of experimental results and a portion of an electroporation waveform are illustrated, respectively, consistent with the present inventive concepts. FIG. 4 shows example experimental results providing total sinusoidal cycle length across the x-axis and microbubble formation measured in nanolitres along the y-axis. Under each cycle length regime, four measurements were taken with an average microbubble formation volume as indicated by the bars of the figures, and the accompanying numbers. The error on each measurement is provided by the presented error bars. The cycle length, in these cases, is directly proportional to the inter-pulse delay period 220, as the energy pulse 210 duration is not changed between cycle lengths in this experiment. As can be seen from the figure, microbubble formation is reduced by more than a factor of 5 when moving from a low (0.5 ms to 5 ms) to a moderate (5 ms to 30 ms) cycle length. An even greater reduction in microbubble formation is attained when moving to an improved (30 ms to 100 ms) cycle duration.

FIG. 4A shows a portion of an embodiment of electroporation waveform 200, such as electroporation waveform 200 described in reference to FIG. 1 and otherwise herein. Electroporation waveform 200 shown includes two energy pulses 210 separated by an inter-pulse delay period 220. In some embodiments, inter-pulse delay period 220 comprises two portions, fixed inter-pulse delay period 2201 and a variable inter-pulse delay period 2202. For example, the duration of the inter-pulse delay period 220 can comprise the duration of fixed inter-pulse delay period 2201 plus the duration variable inter-pulse delay 2202. The duration of variable inter-pulse delay period 2202 can be a positive and/or a negative duration, such that inter-pulse delay period 220 is longer and/or shorter, respectively, than fixed inter-pulse delay period 2201. The variable inter-pulse delay period 2202 can be limited to a value that varies symmetrically about the fixed inter-pulse delay period 2201, where the result integrated value over time equals the value of the fixed inter-pulse delay period 2201. For example, a first inter-pulse delay period 220 (e.g., a delay between a first energy pulse 210 and a second energy pulse 210) can comprise a duration of 35 ms with a fixed inter-pulse delay period 2201 of 30 ms and a first variable inter-pulse delay period 2202 of +5ms. A second, subsequent inter-pulse delay period 220 (e.g., a delay between the second energy pulse 210 and a third energy pulse 210) can comprise a duration of 25 ms with the fixed inter-pulse delay period 2201 of 30 ms and a second variable inter-pulse delay period 2202 of −5 ms. In this example, the first and second variable inter-pulse delay periods 2202 are symmetric about 0 (+/−5ms, respectively), such that the average inter-pulse delay period 220 between the first, second, and third energy pulses 210 is equal to the fixed inter-pulse delay period, 30 ms, in this example. In other words, in this example, the first variable inter-pulse delivery period 2202 can comprise a duration that is equal to the absolute value of the duration of the second variable inter-pulse delay period. The variable inter-pulse delay period 2202 can be varied each period by an amount that can be either fixed or variable, such as a pseudo-random value (e.g., a pseudo-random value calculated by algorithm 25).

A first inter-pulse delay period 220 between a first energy pulse 210 and a second, subsequent, energy pulse 210 can be different from a second inter-pulse delay period 220 between the second energy pulse 210 and a third energy pulse 210 that follows the second energy pulse 210. That is, inter-pulse delay periods 220 can vary in duration from cycle to cycle 230. In some embodiments, a plurality of inter-pulse delay periods 220 in the electroporation waveform 200 can comprise different inter-pulse delay periods 220 from one-another. In yet other embodiments, each inter-pulse delay period 220 in the electroporation waveform 200 can have a different inter-pulse delay period 220 than each other inter-pulse delay period 220.

In some examples, the duration of each successive inter-pulse delay period 220 is different to its immediately preceding inter-pulse delay period 220 by a fixed period. It will be appreciated that the fixed duration can be a positive or negative duration such that successive inter-pulse delay periods 220 can steadily become longer or shorter. That is, the inter-pulse delay period 220 can increase or decrease in a stepwise arrangement. This configuration may begin from a nominal inter-pulse delay period 220 and vary from that point. The nominal inter-pulse delay period 220 can be any inter-pulse delay period 220 within the range of possible inter-pulse delay periods 220. Further, one electroporation waveform 200 can be followed by another electroporation waveform 200 and successive electroporation waveforms 200 can comprise different inter-pulse delay periods 220 such that, for example, a first set of successive inter-pulse delay periods 220 of a first electroporation waveform 200 can comprise incrementally increasing inter-pulse delay periods 220 followed by a second set of successive inter-pulse delay periods 220 of a second electroporation waveform which comprise incrementally decreasing inter-pulse delay periods 220. As such, over time, a series of electroporation waveforms 200 can provide sets of energy pulses 210 separated by inter-pulse delay periods 220 that vary per series of electroporation waveforms 200 in order to reduce the impact of harmonic stimulation.

In other embodiments, the duration of each inter-pulse delay period 220 of the electroporation waveform 200 can be based on a pseudo-random number (e.g., as calculated by algorithm 25), where the pseudo-random number is independently selected for each inter-pulse delay period 220. It is possible for the first inter-pulse delay period to have the same duration as a second inter-pulse delay period if the pseudo-random number that is selected happens to be the same. However, the likelihood of multiple selected pseudo-random numbers being the same can be extremely small, depending on the range of the unique numbers which can be selected between in the set of pseudo-random numbers. As such, in many embodiments, it will be highly unlikely for two inter-pulse delay periods 220 in an electroporation waveform 200 to have the same duration and, in particular, extremely unlikely for two consecutive inter-pulse delay periods 220 to have the same duration. In one or more embodiments, the controller can be configured to prevent consecutive delay periods from having a same duration.

In some embodiments, the duration of each inter-pulse delay period 220 can comprise both a fixed inter-pulse delay 2201 period and a variable inter-pulse delay period 2202 where the two delay periods are summed together to give the total inter-pulse delay period 220. In these embodiments, the variable inter-pulse delay period 2202 can be based on a pseudo-random number, as described above, and the fixed inter-pulse delay period 2201 can be a predetermined period that does not change between delay periods. This configuration can provide a simple alternative approach to providing for a delay period that is based on a pseudo-random number.

The pseudo-random number can have a value from −1 to 1 and the variable inter-pulse delay period 2202 can be based on a nominal inter-pulse delay period multiplied by the pseudo-random number. In such embodiments, the nominal inter-pulse delay period is a value that is multiplied by a scaling factor where the scaling factor is the pseudo-random number. In other embodiments, the scaling factor can be a value from 0 to 1 or it can be any other value. Using a pseudo-random number that is a value from −1 to 1 allows for a set of random numbers that vary between two bounds equal to the fixed inter-pulse delay period 2201 plus the variable inter-pulse delay period 2202.

In other examples, there may not be provided a fixed inter-pulse delay period 2201 and, instead, the inter-pulse delay period 220 can be entirely variable based on the pseudo-random number. It will further be appreciated that, while pseudo-random numbers are primarily discussed herein, a true random number can alternatively and equivalently be used. True random numbers can be obtained via white-noise generators or other appropriate means, however, such approaches are generally computationally expensive and can also be undesirable in other ways.

In general, whether each inter-pulse delay period 220 is based on a random number generator, a pseudo-random number generator or stepwise variations in the inter-pulse delay period, there can be a fixed minimum difference between different total cycle lengths, which are dependent on the inter-pulse delay period 220. For example, a cycle length can vary by 30, 40 or 50% from a nominal cycle length value. The minimum difference in cycle lengths can be between 0.3 and 10% of the nominal cycle length. By way of example, where there is an electroporation waveform comprising 16 cycles, with a nominal cycle length of 20 ms, then 16 steps can be used from 15 ms to 25 ms, with a 0.625 interval between different cycle lengths. This configuration can translate to a step size of 3.125% of the nominal cycle length. By way of further example, where there is an electroporation waveform comprising 24 cycles and a nominal cycle length of 20 ms, then 24 steps can be used from 15 ms to 25 ms, with a step size of approximately 2% of the nominal cycle length.

Referring now to FIG. 5, a plot of stimulation strength versus duration is illustrated, consistent with the present inventive concepts. FIG. 5 shows an example plot of stimulation duration, shown along the x-axis, and stimulation strength, shown along the y-axis, where the plotted curve is a strength-duration curve which describes minimum combinations of stimulation duration and stimulation strength required to cause muscular or cardiac stimulation (e.g., stimulation to be avoided). The stimulation strength can be measured as a current, however, the same trend is followed where the stimulation strength is a voltage. Cellular stimulation, whether cardiac or muscular, is a function both of the amplitude of an applied stimulus as well as the duration of time over which the stimulus is applied. The rheobase can be defined as a stimulation strength which causes stimulation at infinite duration, which in practice, is at about 300 milliseconds. The chronaxie is defined as the minimum stimulation duration that produces stimulation at a stimulation strength twice that of the rheobase. Strength-duration pulses above or to the right of the plotted strength-duration curve will produce stimulation, while those below or to the left of the line will not.

For pulse field ablation (PFA), the stimulus duration for efficacy is a tradeoff between field strength and duration. Shorter pulses require greater pulse field strength to achieve similar depths of treatment, so, ideally, the frequencies of the pulsed fields will be chosen to be as low as possible, but above the chronaxie. Multiple repetitions of pulsed field energy in an electroporation waveform at these frequencies should then be able to generate a durable lesion while avoiding muscular stimulation, cardiac stimulation, or both. Simulations performed by Applicant have shown that even for theoretically perfect biphasic square waves below the chronaxie, the inherent non-linearity of the tissue leads to residual charge. If subsequent energy pulses in the electroporation waveform repeat quickly enough, this residual charge can integrate over time and lead to undesired stimulation.

For the purposes of avoiding stimulation of muscular or cardiac tissue, controller 110 of system 10 (e.g., system 10 described with reference to FIG. 1 and otherwise herein) can alternatively or additionally be configured to provide for the below-described charge build-up compensation scheme.

In order to provide for an electroporation waveform 200 (e.g., electroporation waveform 200 described in reference to FIG. 1 and otherwise herein) that provides for charge build-up compensation, controller 110 can be configured to cause the provision of one or more time-spaced energy pulses 210 where each energy pulse 210 is configured to cause electroporation. In one or more embodiments, each energy pulse 210 can be an ablation pulse configured to cause irreversible electroporation which results in ablation of target tissue.

Controller 110 can also be configured to provide signaling configured to cause the provision of one or more compensation signals as part of the electroporation waveform 200. The compensation signals are configured to reduce a build-up of charge at a target treatment location on a patient caused by one or more of the plurality of energy pulses 210. The compensation signal can be configured to reduce the build-up of charge by having a predetermined polarity. The polarity can be an opposing polarity to the polarity of the charge build-up. The opposing polarity can be, for example, a polarity that opposes an average polarity of each energy pulses 210 or an average polarity of a plurality of sequential energy pulses 210.

The average polarity of each energy pulse 210 being non-zero may arise where each energy pulse 210 comprises one or more mono-phasic pulses and, as such, comprises an inherent and predetermined polarity. In other embodiments, the energy pulse 210 can comprise one or more biphasic signals, such as biphasic square wave signals and/or biphasic sinewave signals. The one or more biphasic signals can comprise an average polarity which can result from, for example, an asymmetry in the intensity of the positive and negative portions of the signals. The asymmetry in the bi-phasic energy pulses 210 can result in the build-up of charge at the target tissue. Even in bipolar energy pulses 210 that are perfectly uniform and comprise a net-zero average polarity, charge build up at the target tissue may still occur because the inherent non-linearity of the tissue can lead to residual charge build-up.

Cathodal pulses have a greater chance of causing stimulation of muscular or cardiac tissue than anodal pulses. As such, the compensation signals may, in some embodiments, comprise an anodal pulse in order to counteract the charge build-up resulting from cathodal energy pulses.

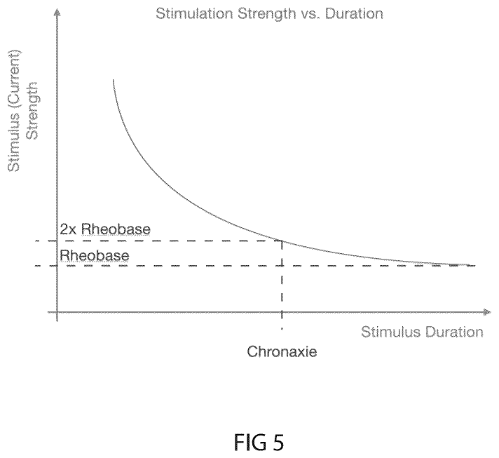

Referring now to FIG. 6, an example of an electroporation waveform comprising compensation signals is illustrated, consistent with the present inventive concepts. FIG. 6 shows an example of an electroporation waveform, such as electroporation waveform 200 of FIG. 1 and otherwise herein. Electroporation waveform 200 can comprise one or more monophasic energy pulses, monophasic energy pulses 2101 (e.g., when energy pulse 210 comprises a monophasic energy pulse 2101), and one or more compensation signals, compensation signals 2102. The compensation signal 2102 can be a monophasic or a biphasic compensation signal. In the case of the monophasic compensation signal 2102, the polarity is determined by whether the signal comprises a positive or a negative amplitude. In the case of a biphasic signal, the polarity of the signal can be defined by whether the signal initially comprises a positive amplitude or a negative amplitude. The frequency and pulse width of the biphasic compensation signal 2102 can be the same, or substantially the same, as the frequency and pulse width of the corresponding energy pulse 210. The amplitude of the compensation signal 2102 can be, for example, at least 1% and/or no more than 20% of the amplitude of the corresponding monophasic energy pulse 2101. Alternatively, the amplitude of the compensation signal 2102 can be, for example, at least 5% and/or no more than 15% of the amplitude of the corresponding monophasic energy pulse 2101. Still further, the amplitude of the compensation signal 2102 can be approximately 10% of the amplitude of the corresponding monophasic energy pulse 2101.

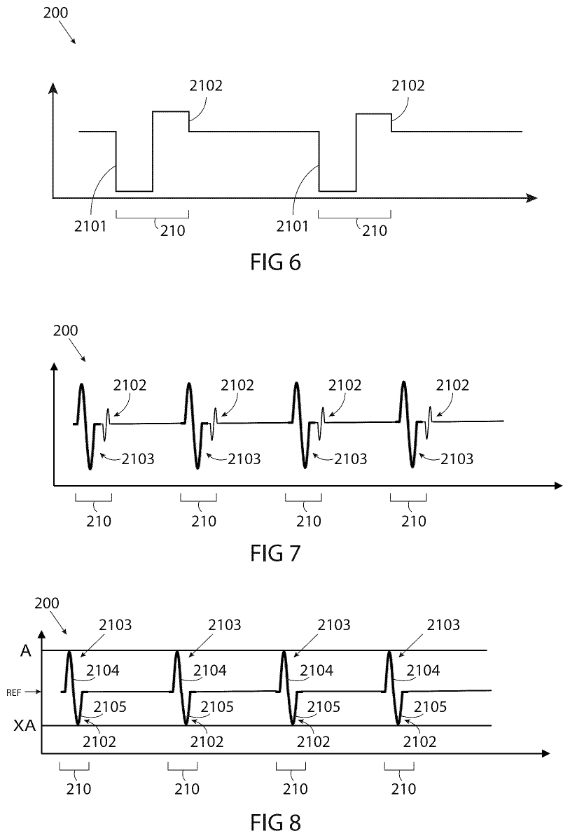

Referring now to FIG. 7, an example of an electroporation waveform comprising compensation signals is illustrated, consistent with the present inventive concepts. FIG. 7 shows an example of an electroporation waveform, such as electroporation waveform 200 of FIG. 1 and otherwise herein. Electroporation waveform 200 can comprise one or more biphasic energy pulses, biphasic energy pulses 2103 (e.g., when energy pulse 210 comprises a biphasic energy pulse 2103), and one or more compensation signals 2102. As can be seen, in this example the polarity of the compensation signals 2102, comprising a biphasic compensation signal 2102, is opposite to that of the biphasic energy pulses 2103.

In some embodiments, compensation signals 2102 comprising monophasic compensation signals 2102 can be provided in an electroporation waveform 200 comprising biphasic energy pulses 2103. The polarity of the compensation signals 2102 can be opposite to that of an average polarity of the biphasic energy pulses 2103. For example, each biphasic energy pulse 2103 can comprise a slight asymmetry in their waveform which results in an average polarity in the electroporation waveform 200 which, in turn, leads to the charge build-up at the treatment location. In other examples, not every biphasic energy pulse 2103 can comprise an asymmetry, or the asymmetry of the biphasic energy pulses 2103 can vary slightly with each pulse, but over time an average polarity of the electroporation waveform 200 can be present which can lead to the charge build-up. In either case, the one or more compensation signals 2102 can be configured to have a polarity opposite to the average polarity of the energy pulses 2101 and/or 2103 of the electroporation waveform 200. The intensity of the compensation signal 2102 can be insufficient to cause ablation of the target tissue and instead is configured to primarily or solely reduce the charge build-up at the target treatment location. The compensation signal 2102 may not provide any therapeutic effect barring the reduction in charge build-up. In particular, the compensation signal 2102 can be configured such that it does not cause electroporation at the target treatment location. By avoiding causing electroporation, such as avoiding irreversible electroporation, the compensation signal parameters will be insufficient to cause cardiac or muscular stimulation by themselves.

The polarity of the compensation signal 2102 can be a “factory-set” polarity. A factory-set polarity can be appropriate, for example, where it is known that a charge build-up will have a particular polarity as a result of a predetermined factor such as the structure of the waveform 200 or the nature of the target tissue. In these embodiments, the factory-set polarity has an opposite polarity to the expected charge build-up. A factory-set polarity will be set at the time of manufacture or at initial configuration of the device. The factor-set polarity of the compensation signal 2102 can either be fixed and unchangeable or it may be adjustable to a different type of polarity, such as a user-set polarity at a later date.

The polarity of the compensation signal 2102 can alternatively be a user-set polarity. This can allow a user to set the polarity of the compensation signal 2102 to a desired value as a result of the user's own observations of a charge build-up. The user can further have control over one or more other parameters of the compensation signal 2102. For example, the user may be able to set the intensity, frequency or linewidth of the compensation signal 2102. Yet further, the user may be able to set a compensation signal delay of the compensation signal 2102 that controls the relative time at which the compensation signal 2102 is provided after the start of the energy pulse 210. It may be most useful to measure the time at which the compensation signal 2102 is provided relative to the start of the energy pulse 210 rather than the end of the energy pulse 210 since, in some embodiments, the compensation signal 2102 may be provided contemporaneously with at least part of the energy pulse 210.

In some embodiments, the polarity of the compensation signal 2102 can be based on a measured charge build-up at the target treatment location. For example, controller 110 can be configured to receive signaling from a charge sensor (e.g., one or more electrodes 311, functional element 199 of generator 100, and/or functional element 399 of catheter 300 comprising and/or configured as a charge sensor) that is configured to detect the charge build-up at the target treatment location, such as on the target tissue or nearby tissue. The charge sensor may be any suitable sensor which is configured to measure a build-up of charge or another parameter which is indicative of the build-up of charge at the target treatment location. The charge sensor may be configured to only detect the polarity of the charge build up or, in other embodiments, may be configured to detect a magnitude of the charge buildup in addition to any other useful parameters. The magnitude of the charge build-up and any other parameters can also be provided to controller 110 in addition to the polarity of the charge build-up. Controller 110 can be configured to set the polarity of the compensation signal 2102 to be opposite to the polarity of the detected charge build-up. Controller 110 can further be able to control one or more other parameters of the compensation signal 2102 based on the signaling received from the charge sensor.

In one or more embodiments, controller 110 can be configured to be switchable between different methods of setting the compensation signal 2102 polarity. For example, controller 110 can be switchable between two or more of setting the polarity based on a factory-set polarity, a user-set polarity and a measured polarity.

At least one compensation signal 2102 can be provided prior to the build-up of charge reaching a predetermined threshold. The predetermined threshold of charge build-up can be the level of charge build-up that results in muscular stimulation when an energy pulse is provided. That is, each energy pulse is configured to have parameters such that neither muscular nor cardiac stimulation is induced absent of charge build-up: each energy pulse 210 is below the chronaxie. The build-up of charge provides a bias to the effective charge experienced by the target tissue when an energy pulse 210 is applied. Thus, the threshold can be based on the minimum current of stimulation minus the stimulation strength (amplitude) of the energy pulse at the duration of stimulation being used.

Controller 110 can be configured to determine (e.g., estimate, calculate, and/or otherwise determine) a total charge build-up based on an expected charge-build up derived from the parameters of the energy pulses 210. Alternatively or additionally, controller 110 can be configured to determine a total charge build-up based on signals received from a charge sensor. The charge sensor (e.g., one or more electrodes and/or functional elements as described herein) can provide charge information to controller 110 via signals representing one or more of the polarity, magnitude, and/or other parameters related to the charge build-up at or near the target tissue at the target treatment area. Thus, controller 110 can be configured to determine the total charge build-up based on one or more sensed parameters related to the charge build-up.

Based on a determined or estimated charge build-up, controller 110 can be configured to provide at least one compensation signal 2102 prior to the build-up of charge reaching the predetermined threshold. For example, a compensation signal 2102 can be provided after an energy pulse 210 in order to reduce the level of charge build-up. Thus, a compensation signal 2102 can be provided after a certain number of energy pulses 210, such as after 5, 10, 20 or 100 pulses 210, by way of example.

In one or more embodiments, controller 110 can be configured to cause the provision of a compensation signal 2102 sequentially after one or more energy pulses 210 or after each energy pulse 210. For example, the compensation signal 2102 can be provided immediately after an energy pulse 210 or after an inter-ablation-compensation delay where an inter-ablation-compensation delay is a period during which no ablation or compensation signal is provided between the provision of these two signals. That is, each compensation signal 2102 can be provided during the delay period between consecutive energy pulses 120. In this example, the compensation signals 2102 may not overlap with any of the energy pulses 210.

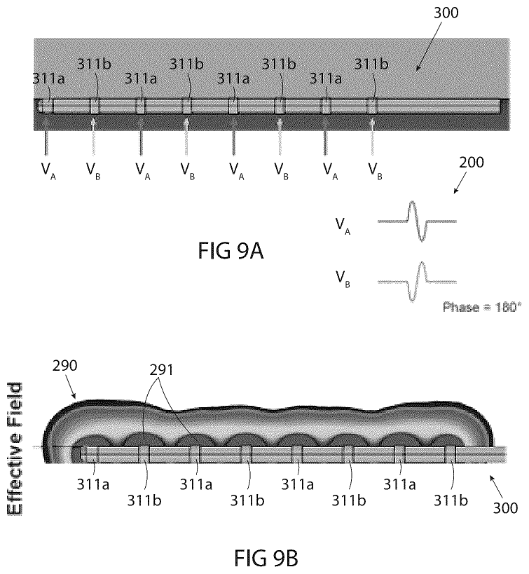

Referring now to FIG. 8, an example of an electroporation waveform comprising a concurrent compensation signal is illustrated, consistent with the present inventive concepts. FIG. 8 shows an example of an electroporation waveform, such as electroporation waveform 200 of FIG. 1 and otherwise herein. In some embodiments, controller 110 is configured to cause the provision of a compensation signal (e.g., compensation signal 2102 described herein) concurrently with at least a portion of one energy pulse 210 (e.g., biphasic energy pulse 2103 shown), a plurality of ablation signals or each ablation signal. In some embodiments, biphasic energy pulse 2103 can include at least one positive portion, positive portion 2104, and/or at least one negative portion, negative portion 2105. In some embodiments, positive portion 2104 comprises a portion of biphasic energy pulse 2103 that is above a reference voltage, REF shown, such as 0V, and/or a reference voltage that is greater than zero, such as a DC offset of biphasic energy pulse 2103, as shown. Similarly, negative portion 2105 can comprise a portion of biphasic energy pulse 2103 that is below the reference voltage REF, such as 0V, and/or a reference voltage that is greater than zero, such as a DC offset of biphasic energy pulse 2103, as shown. In some embodiments, biphasic energy pulse 2103 comprises a signal comprising only positive signals, for example when the DC offset of biphasic energy pulse 2103 is greater than the negative amplitude of negative portion 2105. In some embodiments, compensation signal 2102 may be provided such that it interferes constructively and/or destructively with at least part of a corresponding biphasic energy pulse 2103. For example, a monophasic compensation signal 2102 may be provided such that it interferes destructively with each biphasic energy pulse 2103, in the case of the example depicted in FIG. 8, with a second half of a bi-phasic energy pulse 2103. In FIG. 8 compensation signal 2102 cannot be seen independently, but is destructively interfering with negative portion 2105, such that negative portion 2105 comprises a smaller amplitude than positive portion 2104, as shown (e.g., negative portion 2105 comprises compensation signal 2102 as shown). This configuration results in the apparent amplitude of the negative portion 2105 of the energy pulse 210 having a reduced amplitude compared to the positive portion 2104 of the energy pulse 210. For example, the amplitude of the positive portion 2104 of the biphasic energy pulse 2103 can have an amplitude of “A” (e.g., the absolute amplitude of positive portion 2104 relative to the reference voltage of biphasic energy pulse 2103) while the apparent intensity of the negative portion 2105 of biphasic energy pulse 2103 (the negative portion 2105 of the energy pulse 210 destructively interfered with a positive compensation signal 2102) can have an intensity of “XA” (e.g., the apparent intensity of negative portion 2105 relative to the reference voltage of biphasic energy pulse 2103) where X is a number between 0 and 1. For example, XA can be equal to 0.8 A, as shown in the example of FIG. 8. In other examples, the value of X can be greater than 1. This configuration may be the case where the compensation signal 2102 interferes constructively with the negative portion 2105 of the energy pulse 210 to provide an apparent intensity of greater than amplitude A. It will be appreciated that, in this example, amplitude A is the unadjusted amplitude of the biphasic energy pulse 2103 signal. Correspondingly, the compensation signal 2102 may be provided such that it overlaps with the positive portion 2104 of the energy pulse 210 or such that it overlaps with both the positive and negative portions 2104, 2105 of the energy pulse 210. The phrase “apparent intensity” is used above to refer to a measured intensity of the negative portion of the energy pulse which has undergone destructive interference with the compensation signal. This intensity may differ from the intensity of the negative portion when not experiencing interference and so provide an apparent intensity different to an intensity that is not undergoing interference.

In yet other examples, some compensation signals 2102 may be provided sequentially after a respective one of the energy pulses 210, and other compensation signals 2102 may be provided concurrently with at least a portion of one or more energy pulses 210 of the electroporation waveform 200.

Where the compensation signals 2102 comprise biphasic sinewave signals, the energy pulses 210 can comprise one or more distinct sinewave signals. It will be appreciated that herein the phrase “distinct sinewave signals” is used in order to define sinewave signals which are distinguishable from one-another. This terminology may relate to sinewaves which are entirely discrete from one-another in that they start at a reference voltage, cross through the reference voltage and end at substantially the same reference voltage prior to the next sinewave beginning. The reference voltage can be zero Volts, but it is also possible for the reference voltage to have a different, non-zero value. Distinct sinewaves can also relate to sinewaves which are partially overlapping but in such a manner that the sinusoidal waveform of both sinewaves is distinguishable. Sinewaves which overlap in such a way that they constructively interfere to produce sinewaves of a summed greater amplitude or which form a sinewave or other waveform with a broader period (e.g., linewidth) than any of the original sinewaves, where the constituent sinewaves are not separately distinguishable would not necessarily be considered distinct sinewave signals.

While the build-up of charge at a target treatment location is discussed herein in the context of the configuration of the compensation signal 2102, this discussion is provided to explain the purpose of the compensation signals 2102. It will be appreciated that, based on this goal, the compensation signal 2102 will have certain parameters, such as the polarity and signal intensity which will provide for the desired functionality. The presence of target tissue is not necessary for controller 110 to provide signaling that causes the provision of both the energy pulses 210 and the compensation signals 2102.

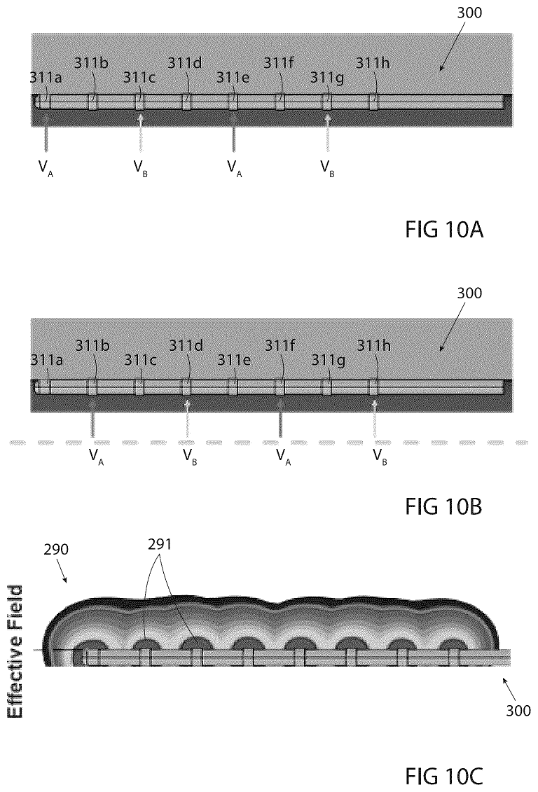

Referring now to FIGS. 9A and 9B, an anatomic side view of a catheter comprising multiple electrodes positioned proximate tissue and a representation of the effective electric field generated by the electrodes are illustrated, respectively, consistent with the present inventive concepts. Catheter 300 and/or other components of system 10 described in reference to FIGS. 9A and 9B can be of similar construction and arrangement to the similar components described in reference to FIG. 1 and otherwise herein. FIGS. 9A and 9B show an example electroporation catheter 300 that comprises a plurality of electrodes 311, such as electrodes 311a and 311b shown, where electrodes 311a and 311b alternate along the length of the distal portion of catheter 300. FIGS. 9A and 9B depict a method of performing electroporation where each of the electrodes 311 of the electroporation catheter 300 have alternating voltages applied thereto (e.g., alternating voltages are applied to electrodes 311a and 311b, as shown) such that electroporation is applied simultaneously along a portion of the electroporation catheter 300. Electric field 290 that is generated with this method is shown in FIG. 9B. This method of performing electroporation can result in several high temperature regions, such as regions 291 shown, centered on each electrode 311 which overlap with each other to provide significant undesirable heating of the local tissue. The generation of high temperature regions 291 can also result in microbubble formation which further causes problems for irreversible electroporation. The high temperature regions 291 herein refer to areas of localized increased heating about an electrode. The presence of microbubbles has the potential to cause various types of problems for patients, such as cerebral micro-embolisms.

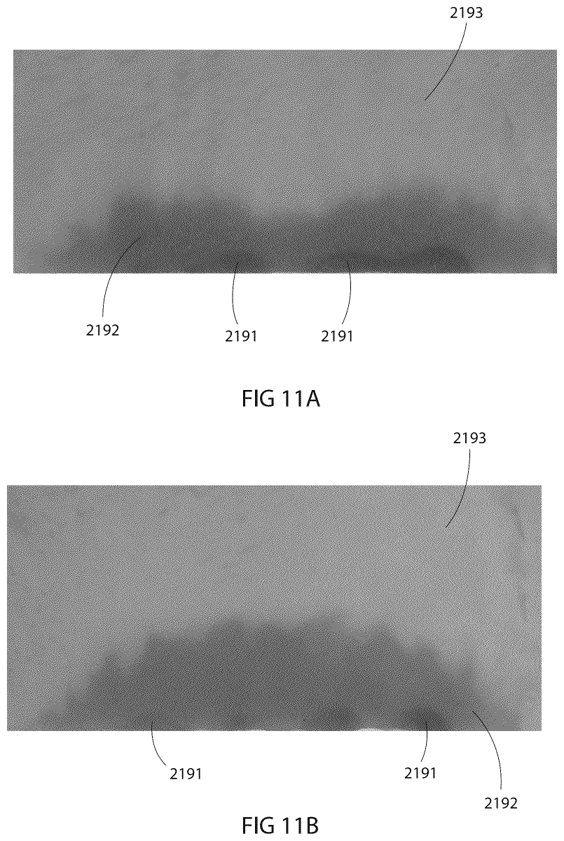

Referring now to FIGS. 10A-C, anatomic side views of a catheter comprising multiple electrodes positioned proximate tissue and a representation of the effective electric field generated by the electrodes are illustrated, respectively, consistent with the present inventive concepts. Catheter 300 and/or other components of system 10 described in reference to FIGS. 10A-C can be of similar construction and arrangement to the similar components described in reference to FIG. 1 and otherwise herein. FIGS. 10A and 10B show electrodes 311 of catheter 300 positioned proximate tissue, for example cardiac tissue. Catheter 300 can be operably attached to a signal generator, such as signal generator 120 of generator 100, not shown but described herein. In some embodiments, system 10 can be configured to operate asynchronously, such as by delivering sequential interleaved bipolar and/or phased-combination energy delivery, as described herein. FIG. 10C shows the aggregate result of the asynchronous energy delivery described herein.

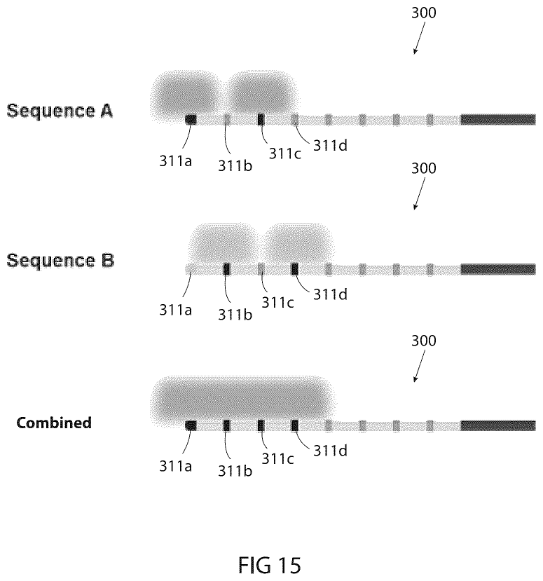

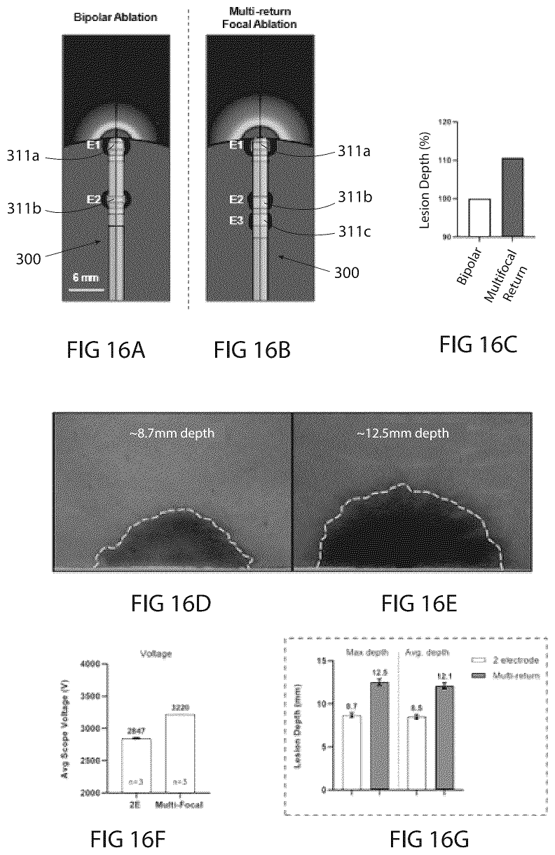

The electroporation catheter 300 comprises a plurality of electrodes 311 arranged consecutively along its length. The electroporation catheter 300 can comprise an elongate member, such as the distal portion of array 310, having electrodes 311 along its length. In some embodiments, the elongate member can have the shape of a cylindrical rod having a circular cross-section or a different shape cross-section. The elongate member can be a straight elongate member or the elongate member can have a curved portion which defines at least a partial arc. Different configurations for the electroporation catheter 300 comprising non-straight elongate members are described in reference to FIG. 13 and FIG. 14 and otherwise herein. In examples where the electroporation catheter 300 comprises an elongate member, whether straight or otherwise, the consecutive arrangement of the electrodes 311 means that the electrodes 311 are arranged one after another such that no two electrodes 311 are provided at the same longitudinal point along the length of the electroporation catheter 300. For example, as shown, electrodes 311 can comprise electrodes 311a-h that are arranged distally to proximally along the distal portion of catheter 300. Electrodes 311a-h can comprise similar or dissimilar spacing, for example equal spacing along catheter 300 as shown (e.g., each electrode 311 is equally spaced from each neighboring electrode). In some embodiments, electrodes 311 comprise a space between each electrode (“electrode spacing”) of no more than 10 mm, such as approximately 6 mm electrode spacing or 3 mm electrode spacing.

In some embodiments, the electroporation catheter 300 can be shaped such that it can comprise a two-dimensional grid of electrodes 311 thereon. That is, a two-dimensional grid of electrodes 311 can comprise a grid of 2×4 electrodes. In such an embodiment, a plurality of electrodes 311 can still be defined to be arranged consecutively or substantially consecutively in an arrangement such that they are arranged one-after another. This consecutive arrangement of electrodes 311 can comprise a subgroup of the total electrodes on the electroporation catheter 300. In the example of a grid of 2×4 electrodes, a single line of electrodes 311 (a 1×4 sub-grid of electrodes of the 2×4 grid) can be considered to be a consecutive arrangement of electrodes 311 according to the present disclosure. By way of another example, a grid of 4×4 electrodes 311 can comprise several sub-grids of electrodes 311 which could be activated in accordance with the present disclosure. It will be appreciated that a one-dimensional grid such as a 1×4 or 1×M arrangement of electrodes 311, where M is an integer equal to or greater than 4, can still be considered as a grid. The requirement for M to be equal to or greater than 4 is explained in further detail below.

In a consecutive arrangement of electrodes, or the grid of electrodes, each electrode 311a-h comprises at least one neighboring electrode 311 in the arrangement. A neighboring electrode 311 comprises herein an electrode 311 which is adjacent to another electrode 311 with no electrode 311 therebetween. For example, in the case of a line of electrodes 311, the electrodes 311 at either end of the line of electrodes 311, such as electrode 311a and 311h, will each have a single neighboring electrode 311, such as electrode 311b and 311g, respectively. Each of the electrodes 311b-g that are not at the end of the line of consecutively arranged electrodes 311 will have two neighboring electrodes 311. In the case of electrodes 311 arranged in a two-dimensional grid, an electrode 311 in a corner of the grid will have two neighboring electrodes 311 (one along a y-axis and one along an x-axis), a non-corner electrode 311 along an edge of the grid will have three neighbors (two along a first axis and one along a second axis) and a non-corner, non-edge electrode 311 will comprise four neighboring electrodes (one on either side of the electrode along each axis). For a given electrode 311 in the arrangement of electrodes 311, any electrode 311 that is not a neighboring electrode may be considered to be a non-neighboring electrode. Electrodes 311 in a two-dimensional grid arranged diagonally from the electrode relative to a consecutive arrangement of electrodes defined by the controller may also be considered to be non-neighboring electrodes.

It will be appreciated that controller 110 can be configured to be able to address electrodes 311a-h individually, and provide signaling to individually control the application of voltages thereto. Further, each of the electrodes 311a-h are suitable for the provision of electroporation to target tissue at a target treatment location. In particular, the electrodes 311a-h are suitable for applying irreversible electroporation to target tissue at a target treatment location. In some embodiments, controller 110 is configured to individually activate and individually send signals to two or more patch electrodes (e.g., an external electrode 60 comprising 2 or more patch electrodes).

As described herein, controller 110 of generator 100 can be configured to provide signaling configured to cause the application of a first potential difference between a set of one or more first electrodes (e.g., a set of one or more electrodes 311 and/or electrodes 60) and a set of one or more second electrodes (e.g., a set of one or more electrodes 311 and/or electrodes 60). In some embodiments, the sets of first and second electrodes comprise any electrode of system 10 configured to delivery energy to tissue, such as any endocardially-positioned electrode (e.g., electrode 311), an electrode of another catheter of system 10, and/or a patient patch comprising an electrode, such as external electrode 60. For example, controller 110 can be configured to provide signaling configured to cause the application of a first potential difference (e.g., energy pulse 210m described herein) between a first electrode 311a and a second electrode 311c of the consecutively arranged plurality of electrodes 311a-h. controller 110 can also be configured to provide signaling configured to cause the application of a second potential difference (e.g., energy pulse 210n described herein) between a third electrode 311b and a fourth electrode 311d of the consecutively arranged plurality of electrodes 311a-h. The first electrode 311a and the second electrode 311c are non-neighboring electrodes. Similarly, the third electrode 311b and the fourth electrode 311d are non-neighboring electrodes. The third electrode 311b is a neighboring electrode to the first electrode 311a. In one or more embodiments, the fourth electrode 311d may similarly be a neighboring electrode to the second electrode 311c, however, in some embodiments, the fourth electrode 311d can be a non-neighboring electrode to the second electrode 311c. That is, the third electrode 311b can be arranged between the first electrode 311a and the second electrode 311c. The second electrode 311c can be arranged between the third electrode 311b and the fourth electrode 311d. It will be appreciated that an electrode 311a-h being arranged between two other electrodes does not necessarily mean that it is the only electrode arranged between these two electrodes, but it does define its general placement relative to the other two electrodes. For example, where the third electrode 311b is arranged between the first electrode 311a and second electrode 311c, a further electrode (such as a fifth electrode) can be arranged directly between the third electrode 311b and the second electrode 311c and, in such an example, the third electrode 311b would still be considered to be between the first electrode 311a and second electrode 311c.

FIG. 10A shows an example of the electroporation catheter 300 where a first potential difference (e.g., electroporation waveform 200) is applied between first electrode 311a and second electrode 311c while no potential difference is applied between third electrode 311b and fourth electrode 311d. In some embodiments, potential differences can also be applied (e.g., applied simultaneously to the first potential difference applied between electrodes 311a and 311c) between non-neighboring electrodes 311e and 311g, while no potential difference is applied between non-neighboring electrodes 311f and 311h, as shown. FIG. 10B shows an example of the electroporation catheter 300 where a second potential difference is applied between the third electrode 311b and fourth electrode 311d while no potential difference is applied between the first electrode 311a and second electrode 311c. In some embodiments, potential differences can also be applied (e.g., applied simultaneously to the second potential difference applied between electrodes 311b and 311d) between non-neighboring electrodes 311f and 311h, while no potential difference is applied between non-neighboring electrodes 311e and 311g, as shown. In some embodiments, the first potential difference is applied asynchronously from the second potential difference. Asynchronously herein is used to explain that the first potential difference is applied over a different and non-overlapping period to application of the second potential difference. It may be preferable to have a potential difference delay period where no potential differences are applied to any of the electrodes 311a-h of the electroporation catheter 300 during the potential difference delay period. This may provide time for the target tissue at the target treatment location to reduce in temperature.

By providing for this asynchronous application of potential differences (e.g., energy pulses 210), the localized heating around individual electrodes 311a-h, high temperature regions 291 shown, is kept more contained than if all of the electrodes 311a-h of the electroporation catheter 300 have potential differences applied therebetween, such as shown in the example of FIG. 9B. FIG. 10C shows the total heating (e.g., high temperature regions 291) and effective electric field 290 that is achieved when providing asynchronous application of potential differences between pairs of non-neighboring electrodes, as described herein. As can be seen when comparing FIG. 9B to FIG. 10C, the undesired heating, high temperature regions 291, is significantly more constrained in extent when using the asynchronous application of potential differences than when applying potential differences between all of the electrodes in the plurality of electrodes.

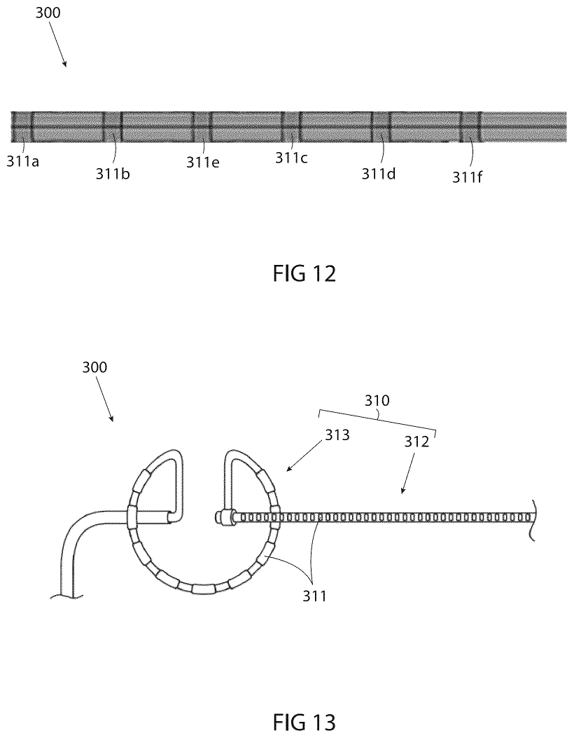

Referring now to FIGS. 11A and 11B, representations of the damage to tissue caused by various forms of electroporation are illustrated, consistent with the present inventive concepts. FIG. 11A shows an example depiction of the resulting tissue damage caused by an electroporation in the mode described with reference to FIGS. 10A and 10B. The darkest regions, region 2191, towards the bottom of FIG. 10A represent undesirable damage caused by heating. The next, middle, region, region 2192, shown in FIG. 11A represents ablated tissue, as is desired during irreversible electroporation. The lightest region, region 2193, represents unaffected tissue (e.g., non-ablated tissue).

FIG. 11B shows an example depiction of the results of controlling an electroporation catheter in the mode described with reference to FIGS. 10A-C. The darkest regions, regions 2191, which only extend slightly from the bottom of the figure represent undesirable damage caused by heating. The next, middle, region, region 2192, shown in FIG. 11B represents ablated tissue, as is desired during irreversible electroporation. The lightest region, region 2193, represents unaffected tissue (e.g., non-ablated tissue). FIG. 11B is provided on the same scale as FIG. 11A and, as such, is directly comparable. As can be seen, the amount of undesirable damage region 2191 resulting from heating is significantly reduced in FIG. 11B compared to the same damage in FIG. 11A. Additionally, the depth of penetration of ablated tissue region 2192 can be increased by providing alternately applied potential differences across interleaved pairs of electrodes 311 as compared to the approach depicted in FIGS. 10A and 10B. As such, the benefits of using this approach are twofold. The provision of alternating activation of the pairs of electrodes 311 can be repeated for as long as desired in order to provide for the desired levels of ablation.

It will be understood that the application of a potential difference between two electrodes (e.g., two electrodes 311) requires the provision of different electrical potential between the two electrodes. As such, a relatively positive electrical potential can be applied at, for example, a first electrode 311 and a relatively negative potential can be applied at a second electrode 311, in order to provide for the first potential difference. While here the electrical potential at a point is defined as relatively positive and negative, these are relative to each other. One may alternatively measure the potential at these points relative to another electrical potential and, in such cases, the potentials can be considered to be respectively: positive and negative; positive and neutral; positive and less positive; negative and neutral; negative and less negative. In any of these cases, there is a difference between the electrical potentials which provides for the potential difference, which is measured in Volts. A potential could be provided between three electrodes (e.g., three electrodes 311), as described in more detail below, such as by applying a same first electrical potential at two electrodes 311 and applying a different electrical potential at another electrode 311 arranged between the two electrodes 311. This configuration would provide for a same first potential difference between two pairs of electrodes 311 where the middle electrode is one of the electrodes in both pairs.

In some embodiments, the first electrode 311a and second electrode 311c (e.g., of FIGS. 10A-C) can be two electrodes of a first subset of electrodes of the plurality of electrodes—311a-h. Similarly, the third electrode 311b and fourth electrode 311d can be two electrodes in a second subset of electrodes of the plurality of electrodes 311a-h. The first subset of electrodes 311 can be interleaved with the second subset of electrodes 311 such that each consecutive electrode in the plurality of electrodes 311a-h alternately belongs to the first subset of electrodes and the second subset of electrodes. That is, each of the electrodes 311 of the first subset of electrodes can be non-neighboring electrodes to each other and each of the electrodes 311 of the second subset of electrodes can be non-neighboring electrodes to each other. In general, the electrodes 311 of any given subset of electrodes 311 can be non-neighboring to each other due to the interleaved arrangement of the electrodes of different electrode subsets. In this example, the number of electrodes 311 in the second subset of electrodes comprises from n−1 electrodes to n+1 electrodes, where n is the number of electrodes in the first subset of electrodes. It will be appreciated that, from the above description, the total number of electrodes 311 in the second subset of electrodes 311 must be equal to at least two, as the second subset of electrodes 311 comprises at least the third and fourth electrodes. For example, where there are three electrodes 311 in the first subset of electrodes, there could be two electrodes 311 in the second subset of electrodes that each have electrodes 311 from the first subset of electrodes on each side of them. Alternatively, there can be up to five electrodes 311 in the second subset of electrodes where electrodes from the second subset of electrodes are located on either side of each of the electrodes of the first subset of electrodes.

In embodiments where the first subset of electrodes 311 comprises more than two electrodes 311, then the first potential difference can be applied between each subset-adjacent electrode of the first subset of electrodes. A subset-adjacent electrode of a given subset to a given electrode can be the next electrode along a consecutive arrangement of electrodes that is part of the same subset of electrodes. For example, the second electrode 311c is a subset-adjacent electrode of the first electrode 311a. A fifth electrode 31le can also be part of the first subset of electrodes and the fourth electrode 311d can be arranged between the second electrode 311c and the fifth electrode 311e. In this embodiment, the fifth electrode 311e can also be a subset-adjacent electrode to the second electrode 311c but not to the first electrode 311a. In this example, the first electrode 31la and fifth electrode 31 le can be provided with a relatively positive electrical potential while the second electrode 311c can be provided with a relatively negative voltage such that potential differences are provided between the first electrode 31 la and second electrode 311c and between the second electrode 311c and fifth electrode 31 le. Similarly, the second subset of electrodes can further comprise a sixth electrode 311f where the fifth electrode 311e is arranged between the fourth electrode 311d and sixth electrode 311f. A second potential can be applied between the third electrode 311b and fourth electrodes 311d and between the fourth electrode 311d and sixth electrode 311f by applying a relatively positive electrical potential at the third electrode 311b and sixth electrode 311f and by applying a relatively negative potential at the fourth electrode 311d.

It will be appreciated that the first potential difference and the second potential difference can be the same potential difference. Alternatively, the first potential difference and the second potential difference can be different to each other. In embodiments where multiple potential differences are provided between three or more electrodes 311 of a given subset of electrodes, the potential differences applied between adjacent electrode pairs can be the same or may be different. For example, it may be desirable to obtain uniform ablation across at least a portion of the length of the electroporation catheter 300 (e.g., along the length of electrode array 310). In other examples, it may be desirable to increase the ablation, or depth of ablation, at certain points along the length of the electroporation catheter 300.

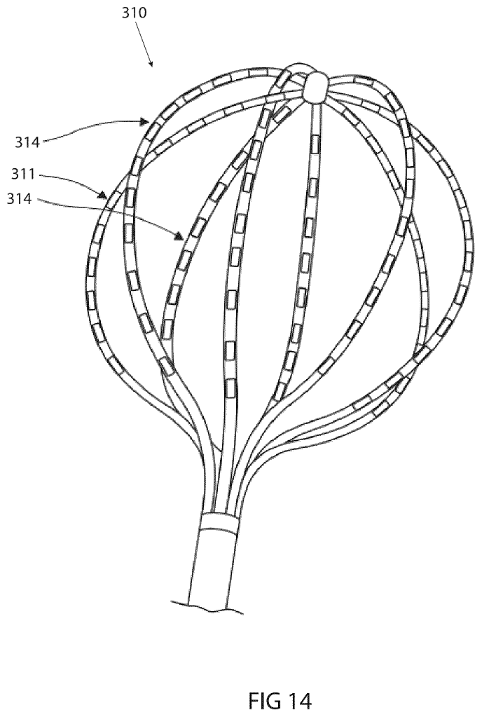

Referring now to FIG. 12, a side view of a catheter including a plurality of electrodes is illustrated, consistent with the present inventive concepts. FIG. 12 shows an embodiment of an electroporation catheter 300 comprising a plurality of electrodes, electrodes 311a-f shown, on which three pairs of electrodes 311 may be defined. In some embodiments, such as that shown in FIG. 12, controller 110 can further be configured to provide signaling to cause the application of a third potential difference between a fifth electrode 311e and a sixth electrode 311f of the electroporation catheter 300. The fifth electrode 311e is arranged between the second electrode 311c and the third electrode 311b. The fourth electrode 311d can be arranged between the third electrode 311b and sixth electrode 311f. That is, three pairs of electrodes 311a, c, 311b, d, and 311e, f may be defined such that each of the consecutively arranged electrodes alternately belong to the first pair, the second pair, the third pair and then the first pair again, and so on. This alternating arrangement can be further extended to arrangements where there are three subsets of electrodes 311 where at least one of the subsets comprises more than two electrodes 311 and all of the subsets are interleaved. In such an arrangement, between two electrodes 311 of a given subset there can be at least one electrode 311 from each of the other interleaved subsets of electrodes 311. In particular, for a perfectly interleaved arrangement, between two electrodes 311 of a given subset there can be a single electrode 311 from each of the other interleaved subsets of electrodes 311. This can be extended to a scenario where there are M subsets of electrodes where M is an integer equal to two or more.

Where fifth electrode 31le and sixth electrode 311f form a third pair, or subset, of electrodes 311 across which a third potential difference is applied, controller 110 can be configured to cause the application of the third potential difference asynchronously from the application of both the first potential difference and the second potential difference.

In summary, controller 110 can be configured to define two, three, four or more unique pairs, or subsets, of electrodes 311 and controller 110 can be configured to provide for the alternating application of potential differences across those pairs, or subsets, of electrodes 311. In other embodiments, two, three, four or more subsets of electrodes 311 having at least two electrodes 311 per subset may be defined where a potential difference is applied between each pair of subset-adjacent electrodes 311 in a subset.

Referring now to FIG. 13, a perspective view of a catheter including a curved array of electrodes is illustrated, consistent with the present inventive concepts. FIG. 13 shows an example electroporation catheter 300 which comprises an elongate member, as previously described. In this example, electrode array 310 of the electroporation catheter 300 comprises a substantially straight portion, linear array 312 shown, having a plurality of electrodes 311 therealong and a curved portion, curved array 313 shown, also having a plurality of electrodes 311 therealong. The electrodes 311 of linear array 312 and those of curved array 313 can be controlled as a single plurality of electrodes 311 such that electrodes 311 of linear array 312 and curved array 313 belong to one of a first subset of electrodes 311, a second subset of electrodes 311, or a higher order-subset of electrodes 311, as described above. Alternatively, the electrodes 311 of linear array 312 and curved array 313 can be configured to be controlled separately such that the electrodes 311 of curved array 313 can be controlled using different parameters to those used to control the electrodes 311 of linear array 312 of the electroporation catheter 300. In either case, electrodes 311 on an electroporation catheter 300 herein can be controlled to provide alternating potential differences between electrodes in interleaved subsets of electrodes as described hereinbefore.

Referring now to FIG. 14, a perspective view of a catheter including an expandable array of electrodes is illustrated, consistent with the present inventive concepts. FIG. 14 shows an example electroporation catheter 300 where electrode array 310 comprises an expandable array including a plurality of elongate members, such as eight arms 314 shown, where each arm 314 comprises a plurality of consecutively arranged electrodes 311. An electroporation catheter 300 having this structure may be referred to as a balloon catheter. Arms 314 of electrode array 310 of the electroporation catheter 300 can be flexible such that they can be pulled to a straight configuration or pushed such that the middle of each arm 314 deflects away from a central axis that extends between the ends of arms 314. In such an embodiment, each arm 314 can be controlled as a separate electroporation catheter 300 comprising a separate set of consecutively arranged electrodes 311. controller 110 can be configured to control each set of electrodes 311 arranged along each arm 314. Each set of electrodes 311 along its respective arm 314 can be controlled to provide alternating potential differences between electrodes in interleaved subsets of electrodes as described hereinbefore.

Referring now to FIG. 15, a visual representation of a method of delivering electroporation therapy is illustrated, consistent with the present inventive concepts. FIG. 15 shows an example method of controlling an electroporation catheter 300 according to the present disclosure. In some embodiments, a first potential difference (e.g., energy pulse 210m described herein) is applied between the non-neighboring first electrode 311a and a second electrode 311c. A second potential difference (e.g., energy pulse 210n described herein) can be applied between the non-neighboring third electrode 311b and fourth electrode 311d. The potential differences can be applied in an asynchronous manner, whereby the effective field distribution produces a continuous cellular ablation lesion, such as is described in reference to FIGS. 10A-C and otherwise herein.

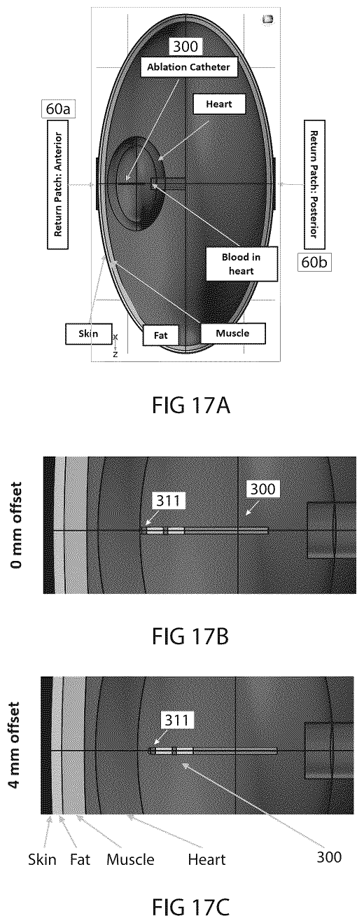

Referring now to FIGS. 16A-G, two anatomic representations and a graph of lesion depth, as well as two representations of damage to tissue and graphs of ablation parameters are illustrated, respectively, consistent with the present inventive concepts. FIGS. 16A and 16B show two electroporation waveform delivery methods. FIG. 16A shows a single return bipolar method of energizing a single electrode, electrode 311a, in combination with a single return electrode, electrode 311b (e.g., providing a potential difference between electrodes 311a and 311b, as shown). FIG. 16B shows a multi-return bipolar method of energizing a single electrode, electrode 311a, in combination with a set of two or more return electrodes, such as electrodes 311b, c shown (e.g., providing a potential difference between electrode 311a and electrodes 311b, c). In some embodiments, catheter 300 comprises a fixed electrode size (e.g., surface area) and spacing. As shown in FIG. 16B, electric field (e.g., field 290) produced with a multi-return method is focused on electrode 311 (e.g., the tip electrode). FIG. 16C shows a graph comparing lesion depth of a lesion created using a single return bipolar method of FIG. 16A and a multi-return bipolar method of FIG. 16B. FIGS. 16D and 16E show examples of lesions created with these two methods, respectively. FIGS. 16F and 16G show additional graphs comparing these two methods of electroporation energy delivery. In the examples shown, the single-return bipolar energy delivery is provided between two similarly sized electrodes, such as two electrodes 311 having approximately a 1:1 ratio of surface area. This single return bipolar ablation method creates a near symmetric electric field distribution between the two poles (e.g., between electrodes 311a and 311b). A multi-return bipolar ablation, which uses multiple return electrodes as described herein, modulates the surface area ratio of ablation while using a fixed catheter configuration (e.g., a fixed catheter 300 geometry), thereby concentrating the electric field 290 at the single electrode (e.g., electrode 311a). The multi-return bipolar ablation configuration of FIG. 16B involves shifting the ratio of surface areas (1:1.1 to 1:1000) to favor a lower total electrode surface area proximate the desired ablation location. This method enhances the lesion and focuses cellular ablation effects to the desired electrode without introducing unipolar electric fields.

Referring now to FIG. 17, a schematic view of a system for performing unipolar and/or phased-combination energy delivery with multiple external patch electrodes is illustrated, consistent with the present inventive concepts. Catheter 300 and/or other components of FIG. 17 can be of similar construction and arrangement to the similar components described in reference to FIG. 1 and otherwise herein. In some embodiments, external electrode 60 can comprise two, three, four, or more patch electrodes, such as external electrodes 60a-e shown. In some embodiments, each of external electrodes 60a-e are individually selectable (e.g., selectable by controller 110), such that energy delivery can be provided between any one or more of electrodes 311 and any one or more of external electrodes 60. In some embodiments, external electrodes 60 can be positioned on the skin of the patient (e.g., positioned by a clinician or other operator of system 10 before and/or during a clinical procedure) in locations selected to enhance the treatment provided by system 10 for various portions of tissue to be treated. For example, an energy delivery can be provided by generator 100 between an electrode 311 and an external electrode 60 that is located in a direction that is generally opposite the intended target tissue, such as when external electrode 60e, located on the back of the patient, is selected for unipolar and/or phased-combination energy delivery when ablating the posterior wall of the heart. In some embodiments, external electrodes 60 are positioned relatively near the heart of the patient (or other target tissue to be treated), such as on the upper back, sides, and/or chest of the patient. In some embodiments, controller 110 is configured to automatically and/or semi-automatically select one or more particular external electrodes 60 to be used as a unipolar return electrode based on the location of the target tissue to be treated. Additionally or alternatively, controller 110 can be configured to select an external electrode 60 based on a measured value, such as a measured impedance between an electrode 311 and each external electrode 60, where optimal one or more external electrodes 60 is selected based on the measurement.

Referring additionally to FIGS. 17A-E, sectional views of the setup of a finite element analysis and the results of the analysis are illustrated, consistent with the present inventive concepts. FIG. 17A shows a model of the body used for a finite element analysis performed by the applicant to quantify the effect of external electrode selection when performing unipolar energy deliveries. The model includes an anterior external electrode, external electrode 60a, and a posterior external electrode, external electrode 60b. The heart is modeled closer to the anterior external electrode 60a, and electrode 311 of catheter 300 is positioned such that the target tissue to be modeled is positioned between electrode 311 and anterior external electrode 60a, as shown. FIG. 17B shows a zoomed in view of FIG. 17A with the model in a first configuration, where electrode 311 is in contact with the target tissue (e.g., positioned with a 0 mm offset). FIG. 17C shows a zoomed in view of FIG. 17A with the model in a second configuration, where electrode 311 is positioned with a 4 mm offset from the target tissue (e.g., not in contact with the target tissue). FIG. 17D shows the result of the finite element analysis, where a 2250V unipolar signal was modeled being delivered between anterior external electrode 60a and electrode 311 positioned with a 0 mm offset from the cardiac tissue and with a 4 mm offset from the cardiac tissue. FIG. 17E shows the result of the finite element analysis, where a 2250V unipolar signal was modeled being delivered between posterior external electrode 60b and electrode 311 positioned with a 0 mm offset from the cardiac tissue and with a 4 mm offset from the cardiac tissue. As shown, when the target tissue is positioned between external electrode 60 (e.g., an anterior-placed external electrode 60a) and electrode 311, the model showed increased ablation of the cardiac tissue for both the 0 mm and 4 mm offset models relative to the similar energy delivery between electrode 311 and posterior external electrode 60b (e.g., where the target tissue is not positioned between the two electrodes). The modeling showed a maximum lesion depth of 13.26 mm when using anterior-placed external electrode 60a, and a maximum lesion depth of 4.77 mm when using posterior external electrode 60b. In vivo testing performed by the applicant resulted in a lesion depth of 15.32 mm (+/−5.18 mm) when a 2080V unipolar signal was provided between electrode 311 (anterior facing) and anterior external electrode 60a. In vivo testing also resulted in a lesion depth of 4.94 mm (+/−0.23 mm) when a 2460V unipolar signal was provided between electrode 311 (anterior facing) and posterior external electrode 60b.

Referring now to FIGS. 18A-C, a graph of a cell membrane potential during an action potential, and examples of various pulse timing methodologies are illustrated, respectively, consistent with the present inventive concepts. Electroporation waveform 200 and/or other components of FIGS. 18A-C can be of similar construction and arrangement to the similar components described in reference to FIG. 1 and otherwise herein. In some embodiments, electroporation waveform 200 is configured to provide, when delivered to tissue, coherent sine-burst electroporation (CSE), for example as described in reference to FIG. 1. Electric fields resulting from CSE can raise the cell membrane potential. In excitable tissue, such as neurons or skeletal muscle tissue, action potentials (APs) can be generated as a result of CSE pulses (e.g., electroporation waveform 200) being delivered. In some embodiments, APs integrate over time and can cause violent muscle contractions. FIG. 18A shows a graph of the membrane potential of a cell over time as a stimulus, such as a CSE pulse, triggers an AP. As shown, the absolute refractory period associated with these APs is approximately 1-2 ms. This absolute refractory period is much shorter than the absolute refractory period of cardiac tissue, which is approximately 250 ms.

As described herein, system 10 can be configured to deliver electroporation waveform 200 (e.g., CSE) using a sequential pulsing method, for example where energy pulses 210 are delivered via odd (e.g., non-neighboring) electrodes 311, and even (e.g., non-neighboring) electrodes 311 independently, such as in an alternating manner (e.g., energy pulses 210m are delivered from first electrode 311a and second electrode 311c, followed by energy pulses 210n that are delivered from third electrode 311b and fourth electrode 311d, as described in reference to FIGS. 10A-C and otherwise herein). In some embodiments, the time period between each energy pulse 210 (e.g., the time period between an energy pulse 210m delivered from odd electrodes 311 and an energy pulse 210n delivered from even electrodes 311), such as interleaving offset period 2203, comprises a time that is greater than the absolute refractory period of an AP, such as between 7 ms and 11 ms. As an example, in a treatment comprising 300 cycles comprising 300 odd energy pulses 210m and 300 even energy pulses 210n (600 total energy pulses 210), a total of 600 APs could be generated during the treatment, as the cell membranes have adequate time to refract between pulses. In some cases, the 600 APs integrate to form violent muscle contractions. As described in reference to FIG. 2 and FIG. 3, electroporation waveform 200 can comprise multiple sequences 250 of bursts 240 of energy pulses 210. In some embodiments, a treatment can comprise 5 sequences 250, each comprising 5 bursts 240, each comprising 24 energy pulses 120 (e.g., 12 odd energy pulses 210m and 12 even energy pulses 210n). This treatment could result in 25 muscle contraction events (one event for each burst 240 of each sequence 250), with 24 APs occurring per event. FIG. 18B shows an example of each odd and even energy pulse 210m, n, respectively, delivery resulting in an AP.

In some embodiments, electroporation waveform 200 is configured to reduce muscle excitation, for example when the timing between odd and even energy pulses 210m, n, respectively, is configured to reduce muscle excitation. For example, even energy pulses 210n can follow odd energy pulses 210m by an interleaving offset period 2203 of less than the absolute refractory period of the AP generated by the odd energy pulses 210m, such as an interleaving offset period 2203 of less than 2 ms, such as approximately 500 μs. The period between the even energy pulses 210n and subsequent odd energy pulses 210m can comprise a longer period, such as a period comprising the difference between inter-pulse delay period 220 and interleaving offset period 2203, for example such that the period between odd energy pulses 210m (and between even energy pulses 210n) is equal to inter-pulse delay period 220, and is the same or similar to the timing shown in FIG. 18B, such as between 12 ms and 21.5 ms between even energy pulses 210n and subsequent odd energy pulses 210m (e.g., when the period between odd energy pulses 210m is between 14 ms and 22 ms, as shown). This timing adjustment can result in a 50 percent reduction in muscle excitation events. FIG. 18C shows an example of this adjusted timing with reduced APs being generated.

Referring now to FIGS. 19A-C, diagrams of various energy delivery modalities are illustrated, consistent with the present inventive concepts. Generator 100, catheter 300, and/or other components of FIGS. 19A-C can be of similar construction and arrangement to the similar components described in reference to FIG. 1 and otherwise herein. FIG. 19A illustrates a unipolar energy delivery modality (e.g., as described in reference to FIGS. 3A-D and otherwise herein), where electric pulse 210 comprises a first signal, VE1, comprising a sine wave that is delivered to a first electrode 311a, and a second signal, VE2, comprising a sine wave that delivered to a second electrode 311b. The phase offset between VE1 and VE2 comprises an offset of 0°, and a reference signal is applied to external electrode 60, as shown. When energy is delivered using this unipolar method, the resultant electric field extends primarily in a direction from each electrode 311 towards external electrode 60, as shown.

FIG. 19B illustrates a bipolar energy delivery modality (e.g., as described in reference to FIGS. 3A-D and otherwise herein), where electric pulse 210 comprises a first signal, VE1, comprising a sine wave that is delivered to a first electrode 311a, and a second signal, VE2, comprising a sine wave that is delivered to a second electrode 311b. The signals VE1 and VE2 comprise a phase offset of 180°, and external electrode 60 is not connected (e.g., a reference voltage is not applied to external electrode 60), as shown. When energy is delivered using this bipolar method, the resultant electric field extends primarily between each electrode 311, without an additional component towards external electrode 60, as shown.

FIG. 19C illustrates a phased-combination energy delivery modality (e.g., as described in reference to FIGS. 3A-D and otherwise herein), where electric pulse 210 comprises a first signal, VE1, comprising a sinewave that is delivered to a first electrode 311a, and a second signal, VE2, comprising a sine wave that is delivered to a second electrode 311b. The signals VEI and VE2 can comprise a phase offset of greater than 0° and less than or equal to 180°. A reference signal is applied to external electrode 60, as shown. When energy is delivered using this phased-combination method, the resultant electric field extends primarily between each electrode 311, and secondarily toward external electrode 60 from each electrode 311, as shown. The phase angle between signals VE1 and VE2 can be adjusted to adjust the relative strength of the primary and secondary components of the resultant electric field, for example as described herein.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for delivering electroporation energy to target tissue to be treated, the system comprising:
a generator configured to provide an electroporation waveform, the generator comprising:
a signal generator configured to generate the electroporation waveform; and
a controller configured to provide signaling configured to cause the signal generator to generate the electroporation waveform;
a catheter comprising at least one catheter electrode, wherein the signal generator is configured to provide the electroporation waveform to the at least one catheter electrode;
wherein the electroporation waveform comprises a plurality of energy pulses, and wherein each energy pulse is separated by an inter-pulse delay period.

2.-40. (canceled)

41. The system according to claim 1, wherein the inter-pulse delay period comprises a first delay period and a second delay period, wherein the first delay period comprises a fixed duration between each of the plurality of energy pulses and the second delay period comprises a variable duration between each of the plurality of energy pulses.

42. The system according to claim 41, wherein each variable duration comprises a positive duration, a negative duration, or both.

43. The system according to claim 41, comprising a first inter-pulse delay period between a first energy pulse of the plurality of energy pulses and a second energy pulse of the plurality of energy pulses, and a second inter-pulse delay period between the second energy pulse and a third energy pulse of the plurality of energy pulses, wherein the first inter-pulse delay period comprises a first variable duration and the second inter-pulse delay period comprises a second variable duration.

44. The system according to claim 43, wherein the first variable delay period comprises a positive duration and the second variable delay period comprises a negative duration.

45. The system according to claim 44, wherein the duration of the first variable duration is equal to the absolute value of the duration of the second variable duration.

46. The system according to claim 41, wherein the variable duration comprises duration based on a pseudo-random number.

47. The system according to claim 1, wherein the inter-pulse delay period comprises a variable duration between each energy pulse.

48. The system according to claim 47, wherein the variable duration comprises a duration based on a pseudo-random number.

49. The system according to claim 47, wherein the variable duration is configured to reduce harmonics created by delivery of the energy pulses.

50. The system according to claim 49, wherein the variable duration is configured to reduce the harmonics by at least 10 dB.

51. The system according to claim 1, wherein the inter-pulse delay period comprises a duration of at least 1 ms.

52. The system according to claim 1, wherein the inter-pulse delay period comprises a duration of no more than 2000 ms.
